(19) United States
(12) Patent Application Publication
(10) Pub. No.: US 2024/0105310 A1
(43) Pub. Date: Mar. 28, 2024

(54) SYSTEMS, METHODS, AND DEVICES, FOR IMPLEMENTING INTERVENTION TREATMENT REGIMENS FOR A POPULATION OF SUBJECTS

(71) Applicant: ICAHN SCHOOL OF MEDICINE AT MOUNT SINAI, New York, NY (US)

(72) Inventors: Jonathan M. DEPIERRO, New York, NY (US); Dennis S. CHARNEY, New York, NY (US); Vanshdeep SHARMA, New York, NY (US); Craig L. KATZ, New York, NY (US); Deborah B. MARIN, New York, NY (US)

(21) Appl. No.: 18/261,907

(22) PCT Filed: Jan. 26, 2022
(86) PCT No.: PCT/US2022/013944

§ 371 Date: Jul. 18, 2023

Related U.S. Application Data
(60) Provisional application No. 63/180,360, filed on Apr. 27, 2021.
(60) Provisional application No. 63/141,940, filed on Jan. 26, 2021.

(51) Int. Cl. G16H 20/70; G16H 10/20; G16H 40/67; G16H 50/20; G16H 50/30; G16H 50/70

(52) U.S. Cl. CPC G16H 20/70; G16H 10/20; G16H 40/67; G16H 50/20; G16H 50/30; G16H 50/70

(57) ABSTRACT

Provided are systems, methods, and devices for implementing a corresponding intervention treatment regimen for the respective subject. A corresponding first plurality of responses of the respective subject is received when provided a first questionnaire, which is applied to a set of models to determine a baseline resilience. A corresponding interactive digital journal associated with the respective subject is generated, allowing access to a corresponding intervention treatment regimen. The corresponding interactive digital journal charts a progress through the intervention treatment regimen by capturing a corresponding subjective narration and a corresponding objective feedback based on a corresponding second plurality of responses of the respective subject when provided a second questionnaire during exposure to the respective intervention treatment regimen. The corresponding intervention treatment regimen includes a corresponding plurality of curricula. When the respective subject is deemed to have completed a curriculum, the objective feedback of the corresponding interactive digital journal is updated.

### CROSS-REFERENCE TO RELATED APPLICATION(S)

The present application claims priority to U.S. Provisional Patent Application No. 63/180,360, entitled “Systems and Methods for Providing a Resilience Building Application to Support the Mental Health of Subjects,” filed Apr. 27, 2021, which is hereby incorporated by reference in its entirety. The present application also claims priority to U.S. Provisional Patent Application No. 63/141,940, entitled “Systems and Methods for Providing a Resilience Building Application to Support the Mental Health of Subjects,” filed Jan. 26, 2021, which is hereby incorporated by reference in its entirety.

### TECHNICAL FIELD

The present disclosure relates to systems, methods, and devices for providing a resilience building application to support the mental health of subjects.

### BACKGROUND

The Coronavirus disease (COVID-19) pandemic has resulted in increased strain on health care systems and negative psychological effects on health care workers. This strain anticipated to result in long term negative mental health effects on the population, with health care workers (HCWs) representing a particularly vulnerable group.

For instance, the COVID-19 has resulted in over 41 million infections with a worldwide case fatality ratio of approximately 2.6% as of early November 2020. See World Health Organization, 2020, “Estimating mortality from COVID-19: Scientific brief, 4 Aug. 2020,” print; World Health Organization, 2020, “WHO Coronavirus Disease (COVID-19) Dashboard|WHO Coronavirus Disease (COVID-19) Dashboard,” online, each of which is hereby incorporated by reference in its entirety. Disease spread has been facilitated by a prolonged incubation period and variable symptomatology and severity. As a result, there is a tremendous burden on the healthcare system, with health care workers (HCWs) experiencing increased stress and demand, and increased risk of COVID infection, particularly for patient-facing staff. See Misra-Hebert et al., 2020, “Impact of the COVID-19 Pandemic on Healthcare Workers' Risk of Infection and Outcomes in a Large, Integrated Health System,” J. Gen. Intern. Med., 35(11), pg. 3293, which is hereby incorporated by reference in its entirety. With consideration for the existing issues of burnout and stress within the healthcare population, the additional impact of COVID-19 on HCWs, who represent a vulnerable group during this pandemic, is a growing concern. See Willard-Grace et al., 2019, “Burnout and health care workforce turnover,” Ann. Fam. Med., 17(1), pg. 36; Shreffler et al., 2020 “The impact of COVID-19 on healthcare worker wellness: A scoping review,” West. J. Emerg. Med., 21(5), pg. 1059; Shaukat et al., 2020 “Physical and mental health impacts of COVID-19 on healthcare workers: A scoping review,” International Journal of Emergency Medicine, 13(1); pg. 1, each of which is hereby incorporated by reference in its entirety. Results from a survey of 2,579 frontline HCWs at Mount Sinai Hospital during Spring 2020 showed that 39% of respondents screened positive for COVID-19 related post-traumatic stress disorder, generalized anxiety disorder, or depression. This displays the growing need for additional mental health support amongst HCWs as a result of the pandemic. See Feingold et al., 2021, “Psychological Impact of the COVID-19 Pandemic on Frontline Health Care Workers During the Pandemic Surge in New York City,” Chronic Stress, which is hereby incorporated by reference in its entirety. Moreover, a patient, even in the medical field, will not inherently recognize the particular etiologies and know the proper treatment for each.

In recent years, mobile health applications (MRAs), or “apps,” on smartphones have become ubiquitous tools for personal health management and behavior tracking. See McCarthy, 2019, “One in Five U.S. Adults Use Health Apps, Wearable Trackers.” Gallup, Inc., print; Sama et al., 2014 “An evaluation of mobile health application tools,” JMIR mHealth uHealth, 2(2), pg. 19, each of which is hereby incorporated by reference in its entirety. MHAs can provide individuals with continuous feedback on health status/progress, push notification reminders, and other useful engagement features. These remote, digital capabilities pose a major advantage for smartphones because they provide users with a mechanism to engage with, study, and interpret their health behaviors more regularly and comprehensively than in an average sequence of in-person doctors' appointments or study visits. See Ventola, 2014, “Mobile devices and apps for health care professionals: Uses and benefits,” P T, 39(5), pg. 356; Sawesi et al., 2016, “The Impact of Information Technology on Patient Engagement and Health Behavior Change: A Systematic Review of the Literature,” JMIR Med. Informatics, 4(1), pg. 1. Informed by the literature that has considered the techniques that drive behavior change through MRAs as well as their effectiveness, implementation of MHAs for personal health and behavior management can be fine-tuned and leveraged to become a powerful tool in behavior modification and overall lifestyle improvement. See Milne-Ives et al., 2020, “Mobile apps for health behavior change: Protocol for a systematic review,” JMIR Research Protocols, 9(1), print; Michie et al., 2017, “Developing and evaluating digital interventions to promote behavior change in health and health care: Recommendations resulting from an international workshop,” Journal of Medical Internet Research, 19(6), print; Han et al., 2018, “Effectiveness of mobile health application use to improve health behavior changes: A systematic review of randomized controlled trials,” Healthcare Informatics Research, 24(3), pg. 207; Owen et al., 2015, “mHealth in the Wild: Using Novel Data to Examine the Reach, Use, and Impact of PTSD Coach,” JMIR Ment. Heal., 2(1), pg. 7, each of which is hereby incorporated by reference in its entirety.

Mobile health applications have been rigorously implemented during the COVID-19 pandemic in order to support contact tracing, symptom tracking, and behavioral management for individuals across the globe. These applications have been deployed to support healthcare systems, governments, and communities to reduce the spread of COVID-19 and its negative long-term effects on the stress and wellbeing of individuals. See Whitelaw, et a., 2020, “Applications of digital technology in COVID-19 pandemic planning and response,” The Lancet Digital Health, 2(8), pg. 435; Balcombe et al., 2020, “An Integrated Blueprint for Digital Mental Health Services Amidst COVID-19,” JMIR Ment. Heal., 7(7), pg. 21718; Figueroa et al., “The Need for a Mental Health Technology Revolution in the COVID-19 Pandemic,” Front. Psychiatry, (11), print, each of which is hereby incorporated by reference in its entirety.

A drawback with existing mobile health applications directed to improving resilience is that they fail to take into account for all of the factors that contribute to resilience. While many such factors have been documented in the literature to affect resilience, ways to quantify such factors or otherwise capitalize on them in an application to help improve resilience have not been adequately elucidated.

The information disclosed in this Background section is only for enhancement of understanding of the general background of the invention and should not be taken as an acknowledgement or any form of suggestion that this information forms the prior art already known to a person skilled in the art.

### SUMMARY

Advantageously, the systems, methods, and devices detailed in the present disclosure address the shortcomings in the prior art detailed above.

The present disclosure provides improved systems, methods, and devices that implement a corresponding intervention treatment regimen for a respective subject in order to improve a respective resilience of the respective subject. For instance, in some embodiments, the systems, methods, and devices of the present disclosure address a current and/or an anticipated psychological state of the respective subject for improvement when exposed to the corresponding intervention treatment regimen.

Turning to more specific aspects, an aspect of the present disclosure is directed to providing a computer system. The computer system includes one or more processing units and a memory coupled to at least one processing unit of the one or more processing units. The memory stores at least one program for execution by the at least one processing unit. The at least one program including one or more instructions, which is provided for each respective subject in a plurality of subjects. The plurality of subjects includes more than 100 subjects. The at least one program includes one or more instructions for receiving a respective first alert from a respective client device associated with a respective subject that the respective subject has initiated a corresponding method. The respective first alert is provided in electronic form at the computer system. Accordingly, the corresponding method includes receiving a corresponding first plurality of responses of the respective subject when provided a first questionnaire at a respective first time. The corresponding first plurality of responses is received in electronic form at the respective client device. Moreover, each respective response in the corresponding first plurality of responses is associated with a corresponding value. Furthermore, the corresponding first plurality of responses includes a corresponding first set of responses associated with an ecological mood of the respective subject, a corresponding second set of responses indicative of whether the respective subject exhibits a depressive disorder or an anxiety disorder, a corresponding third set of responses indicative of a stress reaction of the respective subject, a corresponding fourth set of responses signifying a self-reported resilience of the respective subject, a corresponding fifth set of responses indicative of a general wellness state of the respective subject, a corresponding sixth set of responses indicative of whether the respective subject exhibits an alcohol disorder, and a corresponding seventh set of responses that characterizes a state of spirituality of the respective subject. The corresponding method includes applying, at the respective client device or the computer system, the corresponding first plurality of responses to a first set of models, which determines a baseline resilience of the respective subject at the respective first time. The first set of models includes a first cohort-based questionnaire response normalization model. The corresponding method includes generating, for the respective subject, a corresponding interactive digital journal associated with the respective subject, thereby allowing the respective subject access to a corresponding intervention treatment regimen by the corresponding interactive digital journal. The corresponding intervention treatment regimen is designed to improve the resilience of the respective subject. Furthermore, the corresponding interactive digital journal is configured, at least in part, to chart a progress of the respective subject through the intervention treatment regimen by capturing a corresponding subjective narration and a corresponding objective feedback. The corresponding subjective narration is captured in electronic form at the respective client device. Moreover, the corresponding subjective narration is captured by the respective subject of an exposure to the corresponding intervention treatment regimen. Additionally, the corresponding objective feedback based on a corresponding second plurality of responses of the respective subject when provided, in electronic form, a second questionnaire during the exposure to the respective intervention treatment regimen. The corresponding objective feedback is configured to arouse a behavioral change in the respective subject. The corresponding method includes exposing the respective subject to the corresponding intervention treatment regimen. The corresponding intervention treatment regimen includes a corresponding plurality of curricula. Each respective curriculum in the corresponding plurality of curricula is associated with a unique factorization of a respective resilience of the respective subject and. Moreover, each respective curriculum in the corresponding plurality of curricula includes a unique compilation of digital media configured to improve an ability of the respective subject associated with the unique factorization of the respective resilience, and a corresponding set of instructions for the respective subject to interact with reality. The corresponding plurality of curricula includes a first curriculum associated with a cognitive appraisal of the respective subject. Moreover, the corresponding plurality of curricula includes a second curriculum associated with confronting a first locus of fear experienced by the respective subject. Additionally, the corresponding plurality of curricula includes a third curriculum associated with an environmental reality system determined, at least in part, by the respective subject. The corresponding plurality of curricula includes a fourth curriculum associated with a plurality of biological factors of the respective subject. Further, the corresponding plurality of curricular includes a fifth curriculum associated with confronting a second locus of purpose of the respective subject. Accordingly, the at least one program includes one or more instructions updating a respective user profile associated with the respective subject in response to the receiving the respective first alert. The at least one program includes one or more instructions receiving a respective second alert, in electronic form at the computer system, from the respective client device associated with the respective subject that the respective subject has concluded the corresponding method, thereby updating, in accordance with a determination that the respective subject is deemed to have completed a curriculum in the corresponding plurality of curricula, the objective feedback of the corresponding interactive digital journal. Furthermore, the at least one program includes one or more instructions updating the respective user profile associated with the respective subject in response to the receiving the respective second alert. From this, the computer system implements the corresponding intervention treatment regimen for the respective subject.

In some embodiments, the intervention treatment regimen for the respective subject is self-guided by the respective subject.

In some embodiments, the corresponding value is in a range of from 0 to 4, from 0 to 5, or from 0 to 10.

In some embodiments, the corresponding value is categorical.

In some embodiments, the first questionnaire includes a first set of questions indicative of whether the respective subject exhibits a depressive indicator.

In some embodiments, the depressive indicator includes a sleep quality rating of the respective subject, a mood rating of the respective subject, an anxiety rating of the respective subject, or a combination thereof.

In some embodiments, the first questionnaire includes a second set of questions configured to identify a generalized anxiety disorder exhibited by the respective subject.

In some embodiments, the corresponding first plurality of responses includes an eighth set of responses associated with a demographic status of the respective subject.

In some embodiments, the eighth set of responses includes a gender of the respective subject, an age of the respective subject, a race of the respective subject, an employment status of the respective subject, an education status of the respective subject, a military service status of the respective subject, an income status of the respective subject, an adverse childhood experience status of the respective subject, or a combination thereof.

In some embodiments, the first questionnaire is substantially identical to the second questionnaire.

In some embodiments, the second questionnaire is a subset of the first questionnaire.

In some embodiments, the first questionnaire includes from about 20 questions to about 75 questions.

In some embodiments, the applying the corresponding first plurality of responses to the first set of models includes applying each respective response in the corresponding first plurality of responses to a corresponding model in the first set of models.

In some embodiments, the first set of models includes a second Likert scale model.

In some embodiments, the baseline resilience of the respective subject includes an indication of an adverse condition affecting the respective subject.

In some embodiments, the corresponding set of instructions for the respective subject to interact with reality includes a first instruction for the respective subject to input a respective subjective narration by the respective subject.

In some embodiments, the subjective narration by the respective subject of the exposure to the intervention treatment regimen includes a duration of a disruptive event producing adversity, a severity of the disruptive event producing adversity, an outcome of disruptive event producing adversity, or a combination thereof.

In some embodiments, the unique compilation of digital media includes a first set of textual digital media.

In some embodiments, the first set of textual digital media includes a lecture text portion and/or an activity text portion.

In some embodiments, the unique compilation of digital media includes a second set of video digital media.

In some embodiments, the second set of video digital media includes a lecture by a medical practitioner credentialed to improve the ability of the respective subject associated with the unique factorization of the resilience.

In some embodiments, the charting the progress of the respective subject further includes providing a graphical chart. The graphical chart includes a first axis by a first segmentation based on a period of time. Moreover, the graphical chart includes a second axis by a second segmentation based on a respective set of responses provided by the respective subject. Furthermore, the graphical chart includes one or more data plots. Each respective data plot in the one or more data plots represents a corresponding set of responses in a corresponding second plurality of responses of the respective subject when provided the second questionnaire during the period of time. Additionally, each respective data plot in the one or more data plots populates a unique location within an area formed by the first axis and the second axis.

In some embodiments, the first segmentation and/or the second segmentation is a linear segmentation or a logarithmic segmentation.

In some embodiments, an initial time of the period of time is the first time.

In some embodiments, each respective data plot in the one or more data plots is associated with a corresponding graphical icon in accordance with a status of the corresponding set of responses.

In some embodiments, the first curriculum associated with the cognitive appraisal of the respective subject is configured in accordance with a respective life-stage of the respective subject.

In some embodiments, the first curriculum associated with the cognitive appraisal of the respective subject includes determining an intellectual function score of the respective subject.

In some embodiments, the first curriculum associated with the cognitive appraisal of the respective subject includes determining a cognitive flexibility score of the respective subject.

In some embodiments, the second curriculum associated with confronting the first locus of fear experienced by the respective subject includes suppressing a hypothalamic-pituitary-adrenal response of the respective subject.

In some embodiments, the fourth curriculum associated with the plurality of biological factors of the respective subject includes a change in brain size of the respective subject, a change in sensitivity of neural receptors of the respective subject, a change in neurotransmitter synthetic and/or uptake, or a combination thereof.

In some embodiments, the fifth curriculum associated with confronting the second locus of purpose of the respective subject includes determining a first score of an importance of the second locus of the respective subject and/or determining a coping classification of the respective subject.

In some embodiments, the determination the respective subject is deemed to complete the curriculum in the corresponding plurality of curricula includes determining whether one or more instructions in the corresponding set of instructions for the respective subject to interact with reality is deemed complete.

In some embodiments, the computer system performs the receiving the respective first alert for two or more subjects, five or more subjects, fifty or more subjects, one hundred or more subjects, a thousand or more subjects, or fifty thousand or more subjects.

In some embodiments, the respective client device is a mobile phone.

In some embodiments, the mobile phone includes a touch-sensitive display.

In some embodiments, the mobile phone further includes a power system for powering the touch-sensitive display, a processing unit, and one or more intensity sensors that receive a user input.

In some embodiments, the power system includes a rechargeable battery.

In some embodiments, the user input includes a directional user input.

In some embodiments, the user input is for providing a respective response of the respective subject.

Another aspect of the respective disclosure is directed to providing a client device for providing a user interface on a touch-sensitive display. The user interface is utilized for identifying an objective feedback and viewing the objective feedback to treat an etiology for a mental health condition of a subject associated with the client device. Accordingly, the client device includes the touch sensitive display, one or more processing units, and a memory storing one or more programs for executing by the one or more processing units. The one or more programs include instructions for providing the user interface. The user interface includes a first user interface screen. The first user interface screen includes a first affordance region representing an interactive questionnaire. The interactive questionnaire includes a plurality of selectable responses for a corresponding plurality of prompts. Furthermore, the user interface includes a second user interface screen. The second user interface screen includes a second affordance region representing the objective feedback. The second affordance region is configured to display a notification that corresponds to the respective objective feedback.

In some embodiments, the user interface facilitates receiving, in electronic form at the client device, a first plurality of responses of the subject from the interactive questionnaire at a respective first time. Each respective response in the first plurality of responses is associated with a corresponding value. The first plurality of responses includes a first set of responses associated with an ecological mood of the subject, a second set of responses indicative of whether the subject exhibits a depressive disorder or an anxiety disorder, a third set of responses indicative of a stress reaction of the subject, a fourth set of responses signifying a self-reported resilience of the subject, a fifth set of responses indicative of a general wellness state of the subject, a sixth set of responses indicative of whether the subject exhibits an alcohol disorder, and a seventh set of responses that characterizes a state of spirituality of the subject.

In some embodiments, the one or more programs further includes one or more instructions for applying the first plurality of responses to a first set of models, which determines a baseline resilience of the subject at the respective first time. The first set of models includes a first cohort-based questionnaire response normalization model. Furthermore, the one or more programs includes one or more instructions for generating, for the subject, an interactive digital journal associated with the subject, which allows the subject access to an intervention treatment regimen by the interactive digital journal. The intervention treatment regimen is designed to improve the resilience of the subject. Moreover, the interactive digital journal is configured, at least in part, to chart a progress, through the second user interface, of the subject through the intervention treatment regimen relative to the baseline resilience by capturing a subjective narration, in electronic form at the client device, by the subject of an exposure to the intervention treatment regimen, and the objective feedback. The objective feedback is based on a second plurality of responses of the subject when provided, in electronic form, a second questionnaire during the exposure to the intervention treatment regimen. Furthermore, the objective feedback is configured to arouse a behavioral change in the subject.

In some embodiments, the subject to the intervention treatment regimen. The intervention treatment regimen includes a corresponding plurality of curricula. Each curriculum in the plurality of curricula is associated with a unique factorization of a resilience of the subject. Moreover, each curriculum in the corresponding plurality of curricula includes a compilation of digital media configured to improve an ability of the subject associated with the unique factorization of the resilience of the respective subject. Furthermore, each curriculum in the corresponding plurality of curricula includes a set of instructions for the subject to interact with reality. The corresponding plurality of curricula includes a first curriculum associated with a cognitive appraisal of the subject, a second curriculum associated with confronting a first locus of fear experienced by the subject, a third curriculum associated with an environmental reality system determined, at least in part, by the subject, a fourth curriculum associated with a plurality of biological factors of the subject, and a fifth curriculum associated with confronting a second locus of purpose of the subject.

In some embodiments, the intervention treatment regimen for the respective subject is self-guided by the respective subject.

In some embodiments, the corresponding value is in a range of from 0 to 4, from 0 to 5, or from 0 to 10.

In some embodiments, the corresponding value is categorical.

In some embodiments, the interactive questionnaire includes a first set of questions indicative of whether the subject exhibits a depressive indicator.

In some embodiments, the depressive indicator includes a sleep quality rating of the respective subject, a mood rating of the respective subject, an anxiety rating of the respective subject, or a combination thereof.

In some embodiments, the interactive questionnaire includes a second set of questions configured to identify a generalized anxiety disorder exhibited by the respective subject.

In some embodiments, the corresponding first plurality of responses includes an eighth set of responses associated with a demographic status of the respective subject.

In some embodiments, the eighth set of responses includes a gender of the respective subject, an age of the respective subject, a race of the respective subject, an employment status of the respective subject, an education status of the respective subject, a military service status of the respective subject, an income status of the respective subject, an adverse childhood experience status of the respective subject, or a combination thereof.

In some embodiments, the interactive questionnaire is substantially identical to the second questionnaire.

In some embodiments, the second questionnaire is a subset of the interactive questionnaire.

In some embodiments, the interactive questionnaire includes from about 20 questions to about 75 questions.

In some embodiments, the applying the first plurality of responses to the first set of models includes applying each respective response in the first plurality of responses to a corresponding model in the first set of models.

In some embodiments, the first set of models includes a second Likert scale model.

In some embodiments, the baseline resilience of the respective subject includes an indication of an adverse condition affecting the respective subject.

In some embodiments, the corresponding set of instructions for the subject to interact with reality includes a first instruction for the subject to input the subjective narration by the subject.

In some embodiments, the subjective narration by the subject of the exposure to the intervention treatment regimen includes a duration of a disruptive event producing adversity, a severity of the disruptive event producing adversity, an outcome of disruptive event producing adversity, or a combination thereof.

In some embodiments, the compilation of digital media includes a first set of textual digital media.

In some embodiments, the first set of textual digital media includes a lecture text portion and/or an activity text portion.

In some embodiments, the unique compilation of digital media includes a second set of video digital media.

In some embodiments, the second set of video digital media includes a lecture by a medical practitioner credentialed to improve the ability of the subject associated with the unique factorization of the resilience.

In some embodiments, the charting the progress of the respective subject further includes providing a graphical chart. The graphical chart includes a first axis by a first segmentation based on a period of time. Moreover, the graphical chart includes a second axis by a second segmentation based on a respective set of responses provided by the respective subject. Furthermore, the graphical chart includes one or more data plots. Each respective data plot in the one or more data plots represents a corresponding set of responses in a corresponding second plurality of responses of the respective subject when provided the second questionnaire during the period of time. Additionally, each respective data plot in the one or more data plots populates a unique location within an area formed by the first axis and the second axis.

In some embodiments, the first segmentation and/or the second segmentation is a linear segmentation or a logarithmic segmentation.

In some embodiments, an initial time of the period of time is the first time.

In some embodiments, each respective data plot in the one or more data plots is associated with a corresponding graphical icon in accordance with a status of the respective set of responses.

In some embodiments, the first curriculum associated with the cognitive appraisal of the subject is configured in accordance with a respective life-stage of the subject.

In some embodiments, the first curriculum associated with the cognitive appraisal of the subject comprises determining an intellectual function score of the subject.

In some embodiments, the first curriculum associated with the cognitive appraisal of the subject includes determining a cognitive flexibility score of the subject.

In some embodiments, the second curriculum associated with confronting the first locus of fear experienced by the subject includes suppressing a hypothalamic-pituitary-adrenal response of the subject.

In some embodiments, the fourth curriculum associated with the plurality of biological factors of the subject includes a change in brain size of the subject, a change in sensitivity of neural receptors of the subject, a change in neurotransmitter synthetic and/or uptake, or a combination thereof.

In some embodiments, the fifth curriculum associated with confronting the second locus of purpose of the subject includes determining a first score of an importance of the second locus of the subject and/or determining a coping classification of the subject.

In some embodiments, the client device is a mobile phone.

Yet another aspect of the present disclosure is directed to providing a method for treating a respective etiology for a mental health condition. The method is implemented at a server device. The server device includes one or more processing units and a memory coupled to at least one processing unit of the one or more processing units. The memory stores at least one program for execution by the at least one processing unit. For each respective subject in a plurality of subjects, the at least one program includes one or more instructions for performing the method. The plurality of subjects comprises more than 100 subjects. The at least one program includes one or more instructions for determining an identification of a respective etiology of the respective subject by applying a corresponding plurality of responses of the respective subject to a first set of models. Furthermore, the at least one program includes instructions for displaying, on a display of a client device associated with the respective subject or the server device, a progress of the respective subject when exposed to a corresponding intervention treatment regimen for the respective subject. From this, the at least one program visualizes a progress of the respective subject indicated by the corresponding plurality of responses that suggests the respective etiology that should be addressed by the respective subject to improve the mental health condition of the respective subject.

The systems, methods, and devices of the present invention have other features and advantages that will be apparent from, or are set forth in more detail in, the accompanying drawings, which are incorporated herein, and the following Detailed Description, which together serve to explain certain principles of exemplary embodiment of the present invention.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the several views of the drawings.

FIG. 1 illustrates a block diagram illustrating an embodiment of a system for implementing a corresponding intervention treatment regimen for a respective subject, in which dashed boxes and lines indicate optional features, in accordance with an embodiment of the present disclosure;

FIGS. 2A and 2B collectively illustrate a computer system for implementing a corresponding intervention treatment regimen for a respective subject, in accordance with an embodiment of the present disclosure;

FIG. 3 illustrates a client device for implementing a corresponding intervention treatment regimen for a respective subject, in accordance with an embodiment of the present disclosure;

FIGS. 4A, 4B, 4C, 4D, 4E, and 4F collectively provide a flow chart illustrating exemplary methods for implementing a corresponding intervention treatment regimen for a respective subject, in which dashed boxes indicate optional features, in accordance with some embodiments of the present disclosure;

FIG. 5 illustrates a user interface for displaying one or more aspects of a corresponding intervention treatment regimen for a respective subject, in accordance with an embodiment of the present disclosure;

FIG. 6A illustrates a user interface for selecting a first questionnaire, in accordance with an embodiment of the present disclosure;

FIG. 6B illustrates a user interface for selecting a second questionnaire, in accordance with an embodiment of the present disclosure;

FIGS. 7A, 7B, and 7C illustrate a user interface for receiving a corresponding plurality of responses of a respective subject when provided a respective questionnaire, respectively, in accordance with an embodiment of the present disclosure;

FIGS. 7D, 7E, 7F, and 7G illustrate a user interface for capturing a corresponding objective feedback based on a corresponding plurality of responses of a respective subject when provided a respective questionnaire and/or viewing the respective objective feedback to treat a respective etiology for a mental health condition, respectively, in accordance with an embodiment of the present disclosure;

FIGS. 8A, 8B, 8C, and 8D illustrate another user interface for capturing a corresponding objective feedback based on a corresponding plurality of responses of a respective subject when provided a respective questionnaire and/or viewing the respective objective feedback to treat a respective etiology for a mental health condition, respectively, in accordance with an embodiment of the present disclosure;

FIGS. 9A, 9B, 9C, 9D, and 9E illustrate yet another user interface for capturing a corresponding objective feedback based on a corresponding plurality of responses of a respective subject when provided a respective questionnaire and/or viewing the respective objective feedback to treat a respective etiology for a mental health condition, respectively, in accordance with an embodiment of the present disclosure;

FIGS. 10A, 10B, and 10C illustrate yet another user interface for capturing a corresponding objective feedback based on a corresponding plurality of responses of a respective subject when provided a respective questionnaire and/or viewing the respective objective feedback to treat a respective etiology for a mental health condition, respectively, in accordance with an embodiment of the present disclosure; and

FIGS. 11A, 11B, 11C, and 11D illustrate yet another user interface for capturing a corresponding objective feedback based on a corresponding plurality of responses of a respective subject when provided a respective questionnaire and/or viewing the respective objective feedback to treat a respective etiology for a mental health condition, respectively, in accordance with an embodiment of the present disclosure.

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

The present disclosure is directed to providing systems, methods, and devices for implementing a corresponding intervention treatment regimen for a respective subject. A corresponding first plurality of responses of the respective subject is received from a respective client device associated with the respective subject. Moreover, the corresponding first plurality of responses is received when the respective subject is provided a first questionnaire. Accordingly, the corresponding first plurality of responses is applied to a set of models in order to determine a baseline resilience of the respective subject (e.g., before the respective subject is exposed to the intervention treatment regimen). A corresponding interactive digital journal associated with the respective subject is generated for the respective subject. As such, the corresponding interactive digital journal, when visualized using the respective client device, allows access for the respective subject to a corresponding intervention treatment regimen. In this way, the corresponding interactive digital journal charts a progress through the intervention treatment regimen, such as by one or more graphical representations that represent a progress of the respective subject within the intervention treatment regimen. For instance, the corresponding interactive digital journal charts the progress by capturing a corresponding subjective narration (e.g., a digital journal entry authored, at least in part, by the respective subject). Moreover, the corresponding interactive digital journal charts the progress of the respective subject by capturing a corresponding objective feedback that is provided to the respective subject (e.g., visualized on the respective client device). In some embodiments, the corresponding objective feedback is captured based on a corresponding second plurality of responses of the respective subject when provided a second questionnaire during exposure to the respective intervention treatment regimen. For instance, in some embodiments, the second questionnaire includes some or all of a plurality of questions from the first questionnaire. Furthermore, the corresponding intervention treatment regimen includes a corresponding plurality of curricula, in which each respective curriculum in the corresponding plurality of curricula is associated with a unique factorization of the respective resilience of the respective subject. When the respective subject is deemed to have completed a curriculum in the corresponding plurality of curricula, the objective feedback of the corresponding interactive digital journal is updated. Accordingly, the capturing the corresponding subjective narration and the corresponding objective feedback in the corresponding interactive digital journal associated with the respective feedback, the systems, methods, and devices of the present disclosure implementing the corresponding intervention treatment regimen for the respective subject to improve the respective resilience of the respective subject.

Reference will now be made in detail to various embodiments of the present invention(s), examples of which are illustrated in the accompanying drawings and described below. While the invention(s) will be described in conjunction with exemplary embodiments, it will be understood that the present description is not intended to limit the invention(s) to those exemplary embodiments. On the contrary, the invention(s) is/are intended to cover not only the exemplary embodiments, but also various alternatives, modifications, equivalents, and other embodiments, which may be included within the spirit and scope of the invention as defined by the appended claims.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

Moreover, as used herein, the term “dynamically” means an ability to update a program while the program is currently running.

Furthermore, the terms “client, “subject,” and “user” are used interchangeably herein.

Additionally, as used herein, the term “resilience,” used interchangeably with the term “psychological resilience” means an ability of a respective subject to survive and recover from a likelihood of harm that is caused by a disruptive event producing adversity. Said differently, resilience is a measure of the ability to recover or cope with adverse situations. Psychological resilience has been defined as the capacity to “bounce back” and adapt well in the face of stress and trauma. See Southwick et al., 2016, “Why are some individuals more resilient than others: the role of social support,” World Psychiatry 15(1), pp. 77-79; and Southwick and Charney, 2012, The science of mastering life's greatest challenges. Cambridge University Press, each of which is hereby incorporated by reference.

The present disclosure is based upon an appreciation that spiritual and religious beliefs provide an important resilience resource. See, for example Schwalm et al., January 2021, “Is there a relationship between spirituality/religiosity and resilience? A systematic review and meta-analysis of observational studies,” J. Health Psychol., which is hereby incorporated by reference. However, it is recognized that, in the art, to date there has not been common definitional parameters assigned to define the term “spirituality.” The present disclosure, therefore, by necessity, must encompass a number of different definitions for the term “spirituality” used in the art. Common to these definitions of spirituality, is the recognition that spirituality provides a mechanism that is utilized by a subject, where the mechanism includes the elements of (i) seeking and expressing meaning and purpose in life, or (ii) the ways in which the subject experiences connectedness to time, space, the subject, or other subjects, or a combination of these elements. For instance, in some embodiments of the present disclosure “spirituality” is synonymous to religion, where religion is “[a]n organized system of beliefs, practices, and symbols designed to facilitate closeness to the transcendent or the Divine and foster an understanding of one's relationship and responsibilities with others living in community.” See, George et al., 2000, “Spirituality and health: what we know, what we need to know,” J. Soc. Clin. Psychol. 19, pp. 102-116, which is hereby incorporated by reference. However, recognizing that “spirituality” can occur outside the confines of the religion has led to the appreciation that “religion” and “spirituality” are overlapping constructs. See, “U.S. Public becoming less religious,” Center PR Religion and Public Live 2015, which is hereby incorporated by reference. As such, in some embodiments, spirituality is not limited to theistic constructs (belief in a supreme being), and is not limited to religious constructs (including shared customs and practices). In some such embodiments, spirituality encompasses phenomenological constructs (lessons learned from life experiences), existential constructs (searches for meaning and purpose) and even mystical constructs (relationships between the transcendent, interpersonal, and transpersonal). See, McSherry and Cash, 2004, “The language of spirituality: and emerging taxonomy,” Int J. Nurs Stud 41, pp. 151-161, which is hereby incorporated by reference. Spirituality is often viewed as something that is experienced within or outside formal institutional settings and traditions and often includes the assumption that there is more to life than what can be grasped empirically or fully understood. It evokes concerns, compassion, and a sense of connectedness to something greater beyond ourselves. See, Koenig et al., 1998, “Religious coping and health status in medically ill hospitalized older adults,” J Nery Ment Dis 186, pp. 513-521; and Pargament, 1997, The psychology of religion and coping: Theory, research, practice, New York: Guilford Publications, each of which is hereby incorporated by reference.

In some embodiments, “spirituality” is defined in accordance with the U.S. Consensus Committee as “[t]]he aspect of humanity that refers to the way individuals seek and express meaning and purpose and the way they experience their connectedness to the moment, to self, to others, to nature and to the significant or sacred.” See, Puchalski et al., 2009, “Improving the quality of spiritual care as a dimension of palliative care: the report of the Consensus Conference,” J Palliat Med 12, pp. 885-904.

Given the issues with definitional clarity, ascertainment of the spirituality of a subject, that is the state of spirituality of a subject, requires rigorous practices for choosing measures to accomplish the task. In some embodiments, measures disclosed in Koenig's Spirituality and Health Research (2011), which provides a review of measures and salient issues within spirituality and health research, are used to accomplish this task. See, Koenig, 2011, Spirituality and health research: Methods, measurement, statistics and resources, West Conshohocken, PA, Tempelton Press, which is hereby incorporated by reference. In some embodiments, measures disclosed in Selman et al. and their related dimensions and recommendations for measurement use and development are used to ascertain a state of spirituality of a subject. See, Selman et al., 2011, “The measurement of spirituality in palliative care and the content of tools validated cross-culturally: a systematic review,” J Pain Symptom Manage 414, pp. 728-753, which is hereby incorporated by reference. In some embodiments, measures disclosed in Hill et al. and their related dimensions and recommendations for measurement use and development are used to ascertain a state of spirituality of a subject. See, Hill and Hood, eds., Measures of Religiosity, Birmingham, AL: Religious Education Press; 1999, which is hereby incorporated by reference. In some embodiments, the Spirituality Index of Well-Being (SIWB) Scale is used to ascertain a state of spirituality of a subject. See Daaleman et al., 2002, “The Spirituality Index of Well-Being: Development and testing of a new measure,” Journal of Family Practice 51(11), which is hereby incorporated by reference. In some embodiments, any of the constructs, factors, or questionnaires described or referenced in Parian et al. are used to ascertain a state of spirituality of a subject. See, Parsian et al., 2002, “Developing and validating a questionnaire to measure spirituality: a psychometric process,” Global Journal of Health Science 1(1), pp. 2-11, which is hereby incorporated by reference.

Furthermore, when a reference number is given an “i^{th}” denotation, the reference number refers to a generic component, set, or embodiment. For instance, a light source termed “light source 710-i” refers to the i^{th} light source in a plurality of light source.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. Where particular values are described in the application and claims, unless otherwise stated, the term “about” means within an acceptable error range for the particular value. The term “about” can have the meaning as commonly understood by one of ordinary skill in the art. The term “about” can refer to ±10%. The term “about” can refer to ±5%.

Moreover, as used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. In some embodiments, n is between 10,000 and 1×10⁷, between 100,000 and 5×10⁶, or between 500,000 and 1×10⁶. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

Furthermore, when a reference number is given an “i^{th}” denotation, the reference number refers to a generic component, set, or embodiment. For instance, a property termed “property i” refers to the i^{th} property in a set of properties (e.g., a property 112-i in a set of properties 112).

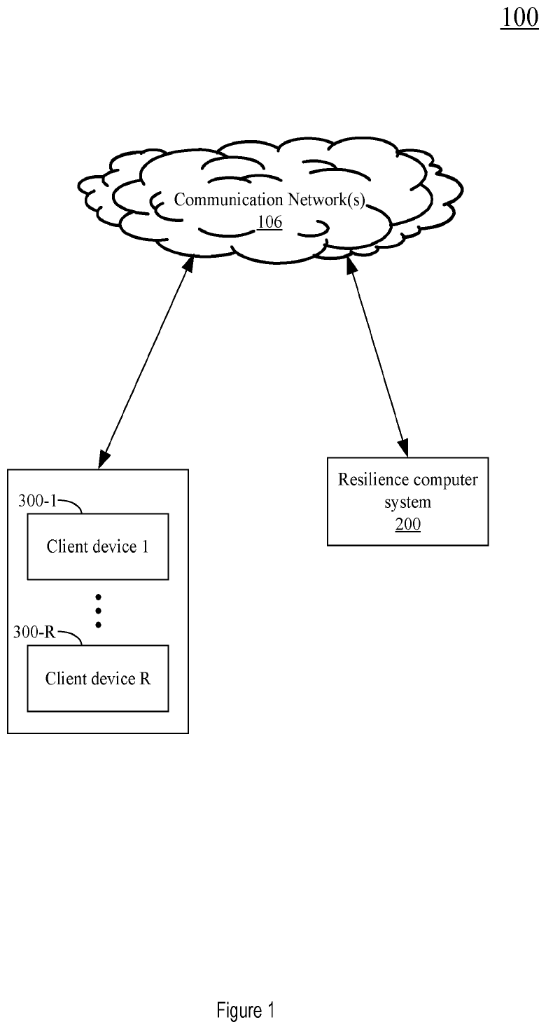

In the present disclosure, unless expressly stated otherwise, descriptions of devices and systems will include implementations of one or more computers. For instance, and for purposes of illustration in FIG. 1, a resilience system 200 is represented as a single device that includes all the functionality of the resilience system 200. However, the present disclosure is not limited thereto. For instance, in some embodiments, the functionality of the resilience system 200 is spread across any number of networked computers and/or reside on each of several networked computers and/or by hosted on one or more virtual machines and/or containers at a remote location accessible across a communications network (e.g., communication network 106 of FIG. 1). One of skill in the art will appreciate that a wide array of different computer topologies is possible for the resilience system 200, and other devices and systems of the preset disclosure, and that all such topologies are within the scope of the present disclosure. Moreover, rather than relying on a physical communications network 106, the illustrated devices and systems may wirelessly transmit information between each other. As such, the exemplary topology shown in FIG. 1 merely serves to describe the features of an embodiment of the present disclosure in a manner that will be readily understood to one of skill in the art.

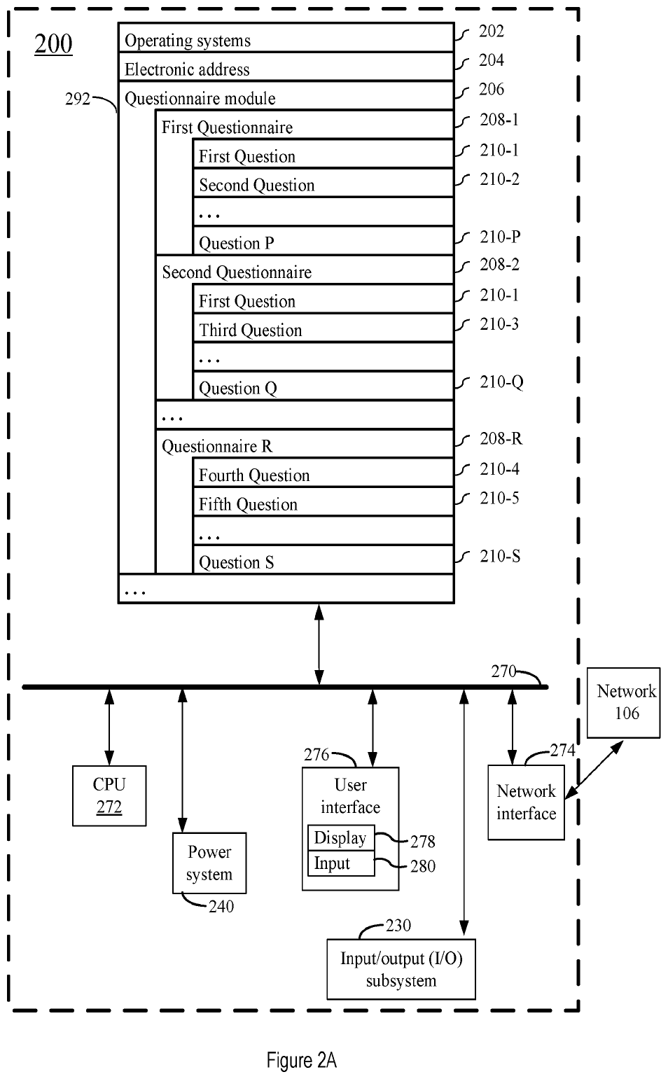

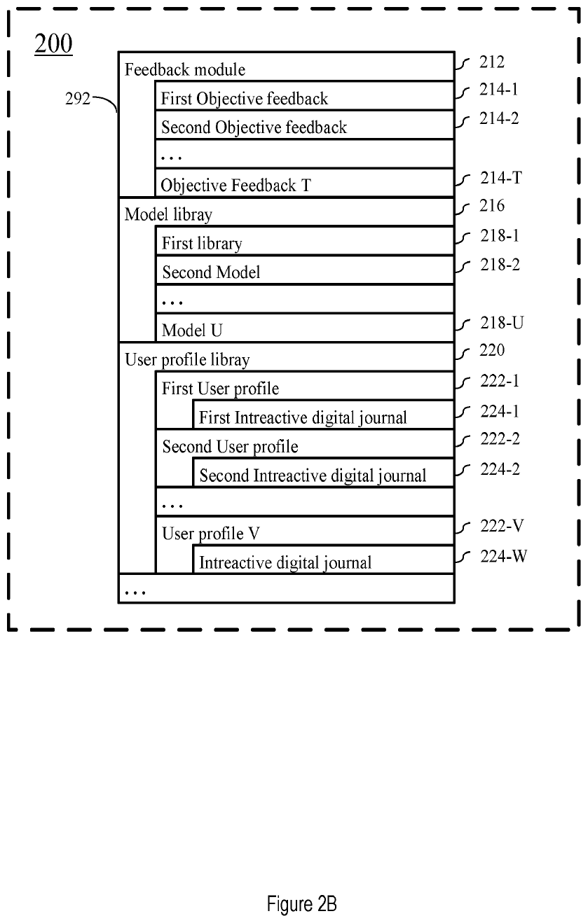

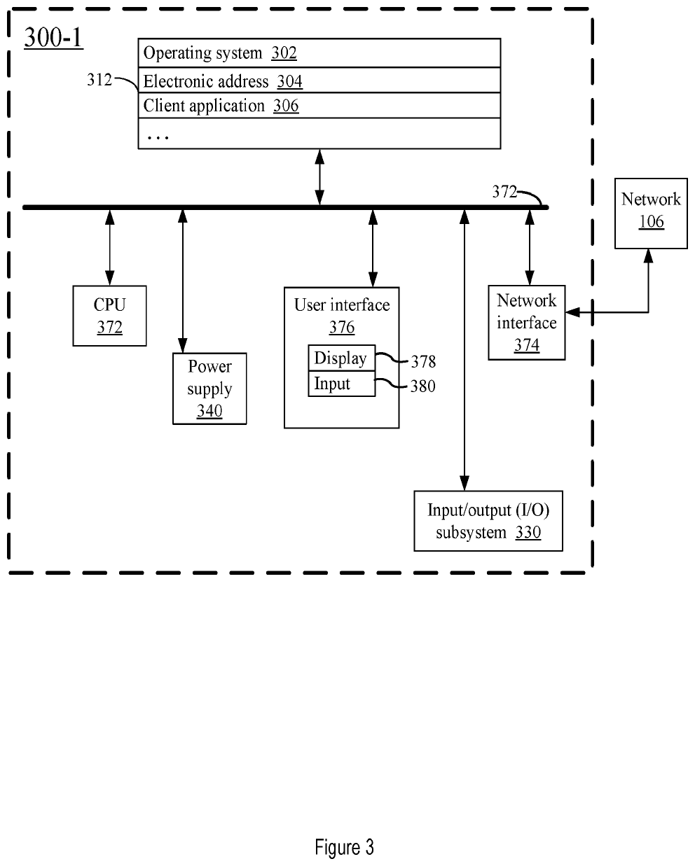

FIG. 1 depicts a block diagram of a distributed computer system (e.g., computer system 100 of FIGS. 2A and 2B and first client device 300-1 of FIG. 3) according to some embodiments of the present disclosure. The computer system 200 at least facilitates identifying one or more combinatorial substitutions that affect one or more properties (e.g., a first property) a target protein.

In some embodiments, the communication network 186 optionally includes the Internet, one or more local area networks (LANs), one or more wide area networks (WANs), other types of networks, or a combination of such networks.

Examples of the network 106 includes, but are not limited to, the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

In some embodiments, the communication network 106 optionally includes the Internet, one or more local area networks (LANs), one or more wide area networks (WANs), other types of networks, or a combination of such networks.

It should be noted that the exemplary topology shown in FIG. 1 merely serves to describe the features of an embodiment of the present disclosure in a manner that will be readily understood to one of skill in the art. Other topologies of the system 100 are possible. For instance, in some embodiments, any of the illustrated devices and systems can, in fact, constitute several computer systems that are linked together in a network or be one or more virtual machines and/or containers in a cloud-computing environment. Moreover, rather than relying on a physical communications network 106, the illustrated devices and systems may wirelessly transmit information between each other.

FIGS. 2A and 2B collectively depict an exemplary resilience computer system 200 for implementing a corresponding intervention treatment regimen for a respective subject in a plurality of subjects. In various embodiments, the resilience computer system 200 includes one or more processing units (CPUs) 272, a network or other communications interface 274, and a memory 292.

The memory 292 includes high-speed random access memory, such as DRAM, SRAM, DDR RAM, or other random access solid state memory devices, and optionally also includes non-volatile memory, such as one or more magnetic disk storage devices, optical disk storage devices, flash memory devices, or other non-volatile solid state storage devices. Memory 292 may optionally include one or more storage devices remotely located from the CPU(s) 272. Memory 212, or alternatively the non-volatile memory device(s) within memory 292, includes a non-transitory computer readable storage medium. Access to memory 292 by other components of the cryptographic node device 220, such as the CPU(s) 272, is, optionally, controlled by a controller. In some embodiments, memory 292 can include mass storage that is remotely located with respect to the CPU(s) 272. In other words, some data stored in memory 292 may in fact be hosted on devices that are external to the resilience system 200, but that can be electronically accessed by the resilience system 200 over an Internet, intranet, or other form of network 106 or electronic cable using communication interface 274.

In some embodiments, the memory 292 of the resilience system 200 for implementing the corresponding intervention treatment regimen for the respective subject stores:
an operating system 202 (e.g., ANDROID, iOS, DARWIN, RTXC, LINUX, UNIX, OS X, WINDOWS, or an embedded operating system such as VxWorks) that includes procedures for handling various basic system services;
an electronic address 204 associated with the resilience system 200 that identifies the resilience system 200;
a questionnaire module 206 that stores a plurality of questionnaires (e.g., first questionnaire 208-1, second questionnaire 208-2, . . . , questionnaire R 208-R of FIG. 2A), each respective questionnaire 208 including a corresponding plurality of questions (e.g., first question 210-1, second question 210-2, question P 210-P of first questionnaire 208-1 of FIG. 2A) configured to illicit one or more responses of the respective subject;
a feedback module 212 for acquiring a respective objective feedback in a plurality of objective feedback (e.g., first objective feedback 214-1 of FIG. 2B), validating the respective objective feedback 214, and providing the respective objective 214 for capture in a corresponding interactive digital journal of the respective subject;
a model library 216 that retains a plurality of models (e.g., first model 218-1, second model 218-2, . . . , model U 116-U of FIG. 2B), each respective model 218 including one or more parameters for tuning a corresponding determination of a respective model 218; and
a user profile library 220 for retaining a plurality of user profiles (e.g., first user profile 222-1, second user profile 222-2, user profile V 222-V of FIG. 2B), each respective user profile 222 associated with a corresponding interactive digital journal that is used to capture a corresponding subjective narration by the respective subject and/or a corresponding objective feedback 214 of the respective subject.

As illustrated in FIG. 2A, a resilience computer system 200 preferably includes an operating system 202 that includes procedures for handling various basic system services. The operating system 202 (e.g., iOS, DARWIN, RTXC, LINUX, UNIX, OS X, WINDOWS, or an embedded operating system such as VxWorks) includes various software components and/or drivers for controlling and managing general system tasks (e.g., memory management, storage device control, power management, etc.) and facilitates communication between various hardware and software components.

In some embodiments, the resilience computer system 200 further includes an electronic address 204 (e.g., a mobile phone number, a social media account, an e-mail address, an internet protocol (IP) address, etc.) that is associated with the resilience computer system 200. In such embodiments, a client application (e.g., client application 306 of client device 300 of FIG. 3) utilizes the electronic address 204 for communication, such as identifying the resilience computer system 200 within a communications network 106. In this way, in some embodiments, the resilience computer system 200 receives specific communications (e.g., responses to a first questionnaire 208) communicated from a client device 300 through the electronic address 204 and the communications network 106.

Referring to FIG. 2B, in some embodiments, the resilience computer system 200 includes a feedback module 212. In some embodiments, the feedback module is utilized for acquiring a respective objective feedback in a plurality of objective feedback (e.g., acquiring first objective feedback 214-1 via communication network 106 for retention in the feedback module 212), validating the respective objective feedback 214 (e.g., determining if the respective objective feedback satisfies a threshold scientific accuracy and/or precision), and providing the respective objective 214 for capture in a corresponding interactive digital journal of the respective subject. However, the present disclosure is not limited thereto.

In some embodiments, the resilience computer system 220 includes a model library 216 that stores a plurality of models 218 (e.g., a first classifier model 218-1, a second regressor model 218-2, a third clustering model 218-3, etc.). In some embodiments, the model library 216 stores two more models 218, three or more models, four or more models, ten or more models, 50 or more models, or 100 or more models. In some embodiments, a respective model 218 in the plurality of models 218 is implemented as an artificial intelligence engine. However, the present disclosure is not limited thereto.

In some embodiments, a user profile library 220 retains a plurality of user profiles 222. Each respective user profile 222 in the plurality of user profiles 222 is associated with a corresponding subject of the resilience computer system. For instance, in some embodiments, the respective subject first customizes his or her profile (e.g., first user profile 222-1) at a client device 300 by providing a plurality of user login information, such as a password, an address (e.g., E-mail address, physical address, etc.), a plurality of demographic information (e.g., age, race, etc.), and the like. In some embodiments, the user profile 222 uniquely identifies the respective user within the resilience computer system 200, such that no two users are associated with a respective user profile 222. In this way, each respective user profile 222 allows the resilience computer system 200 to retain login information, privacy information, geographical data associated with the respective subject (e.g., preferred location(s) for attempting a corresponding set of instructions for the respective subject to interact with reality), biographical data associated with the respective subject, demographic data associated with the respective subject, or a combination thereof.

Each of the above identified modules and applications correspond to a set of executable instructions for performing one or more functions described above and the methods described in the present disclosure. These modules (e.g., sets of instructions) need not be implemented as separate software programs, procedures or modules, and thus various subsets of these modules are, optionally, combined or otherwise re-arranged in various embodiments of the present disclosure. In some embodiments, the memory 292 optionally stores a subset of the modules and data structures identified above. Furthermore, in some embodiments, the memory 292 stores additional modules and data structures not described above.

It should be appreciated that FIGS. 2A and 2B illustrates only an example of the resilience computer system 200, and that the resilience computer system 200 optionally has more or fewer components than shown, optionally combines two or more components, or optionally has a different configuration or arrangement of the components. The various components shown in FIGS. 2A and 2B are implemented in hardware, software, firmware, or a combination thereof, including one or more signal processing and/or application specific integrated circuits. Moreover, in some embodiments, the resilience computer system 200 is a single device that includes all the functionality of the resilience computer system 200. In some embodiments, the resilience computer system 200 is a combination of multiple devices. For instance, in some embodiments, the functionality of the resilience computer system 200 is spread across any number of networked computers and/or reside on each of several networked computers and/or by hosted on one or more virtual machines and/or containers at a remote location accessible across a communications network (e.g., communications network 106, network interface 274, or both). One of skill in the art will appreciate that a wide array of different computer topologies is possible for the resilience computer system 200, and other devices and systems of the preset disclosure, and that all such topologies are within the scope of the present disclosure.

Referring to FIG. 3, an exemplary client device 300 (e.g., first client device 300-1) is provided.

The client device 300 has one or more processing units (CPU's) 202, peripherals interface, memory controller, a network or other communications interface 374, a memory 392 (e.g., random access memory). Furthermore, the client device 300 includes a user interface 376, the user interface 376 including a display 378 and input 380 (e.g., keyboard, keypad, touch screen). In some embodiments, the client device 300 includes optional audio circuitry 472, an optional speaker 460, an optional microphone 462, one or more optional intensity sensors 464 for detecting intensity of contacts on the device 102 (e.g., a touch-sensitive surface such as a touch-sensitive display system 408 of the device 300), optional input/output (I/O) subsystem 330, one or more communication busses 372 for interconnecting the aforementioned components, and a power system 340 for powering the aforementioned components. In some embodiments, the power system 340 includes a rechargeable battery.

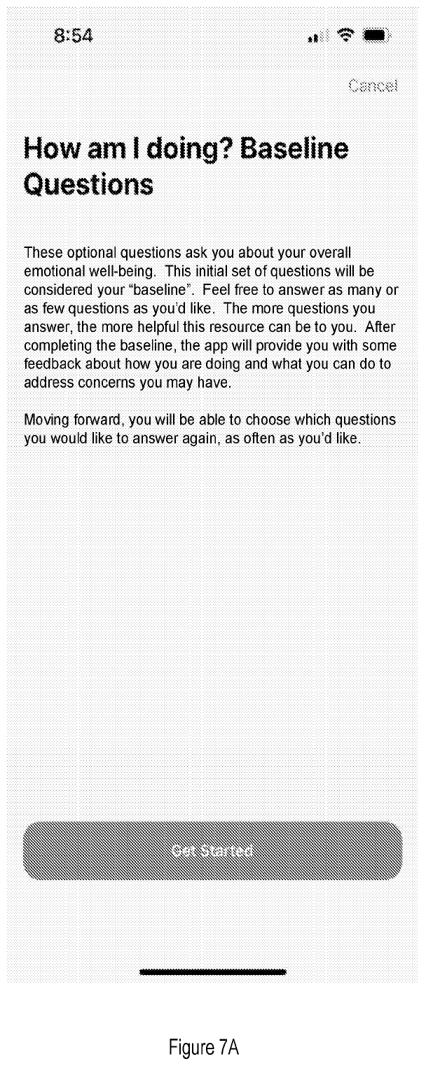

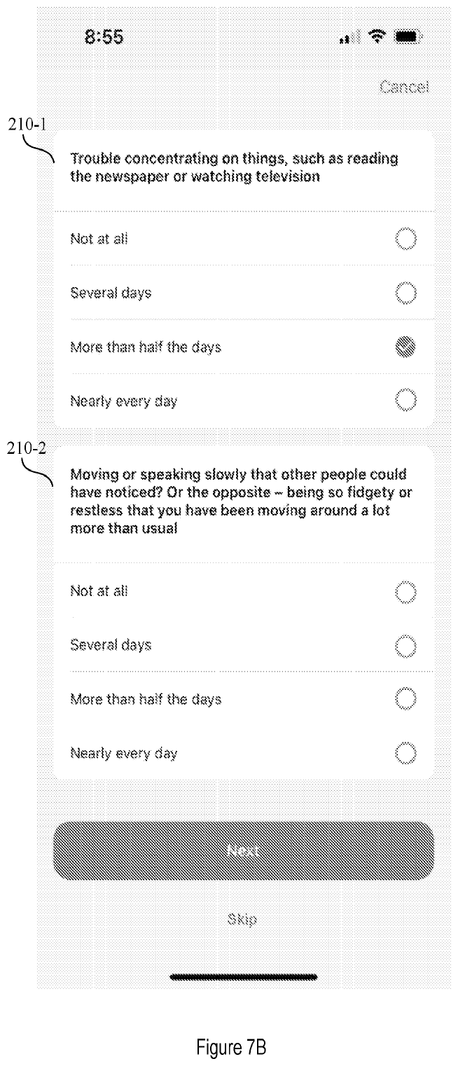

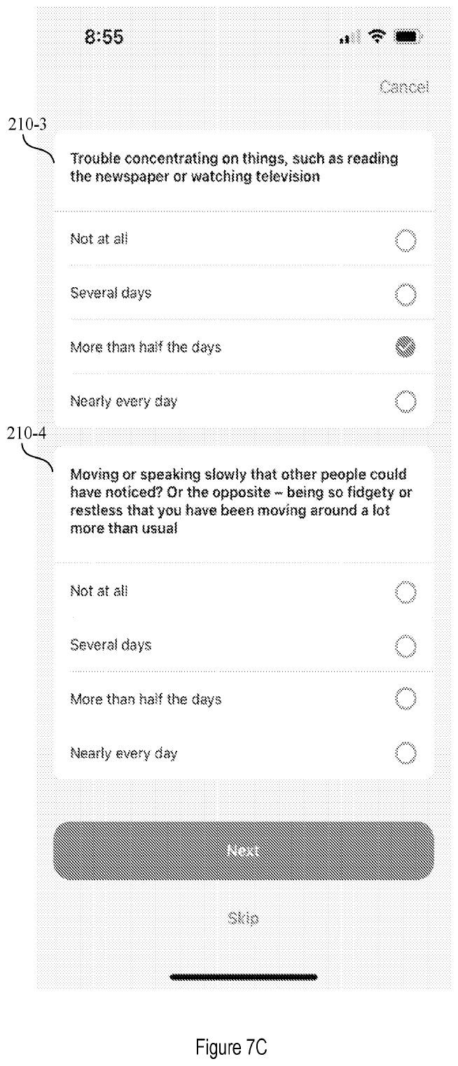

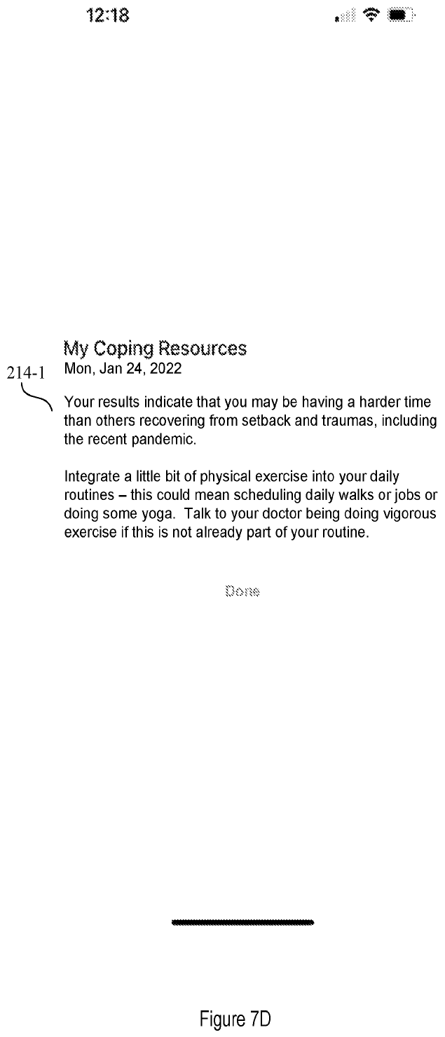

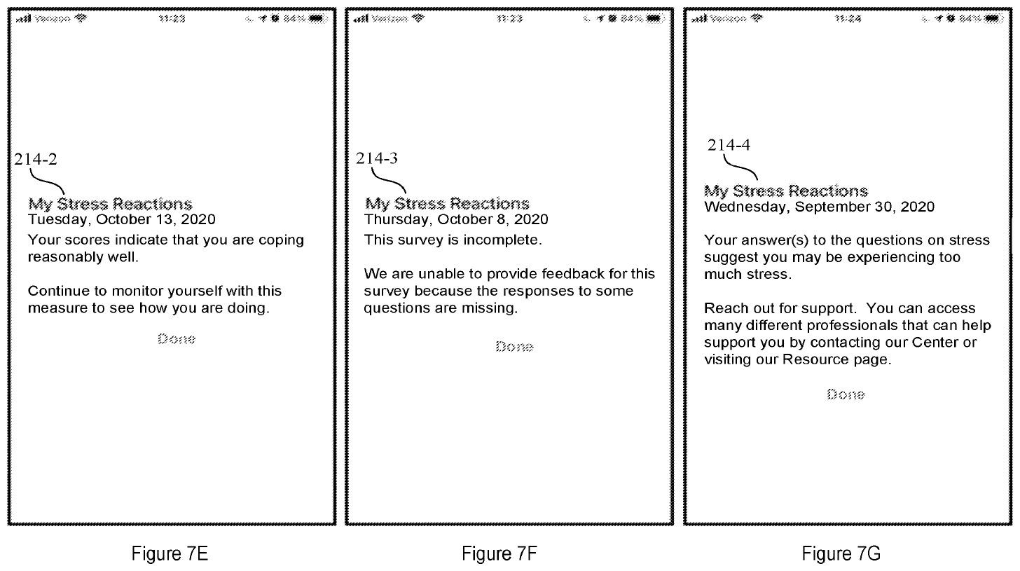

In some embodiments, the input 410 is a touch-sensitive display (e.g., display 408 of FIG. 2), such as a touch-sensitive surface. In some embodiments, the user interface 406 includes one or more soft keyboard embodiments. The soft keyboard embodiments may include standard (QWERTY) and/or non-standard configurations of symbols on the displayed icons. In some embodiments, the computer-enabled imaging device 300 further includes a display 408, and the method further includes displaying information on the display 408, such as one or more boundary conditions (e.g., conditions 624 of FIG. 3) or an image captured by the computer-enabled imaging device 300. In some embodiments, the displayed image is enlargeable or reducible by human touch to the touch screen display 408. In some embodiments, the display 408 is configured for focusing an image of a surface of a region of interest acquired by the two-dimensional pixelated detector. Furthermore, in some embodiments, the display 408 allows for the user to visualize the region of interest when directly viewing the region of interest is undesirable or difficult for the user, such as if the region of interest is exposed to harmful irradiation (e.g., from the one or more light sources 710 of FIG. 7). Additionally, in some embodiments, the display allows the user to visual the region of interest when the region of interest is otherwise out of the line of sight of the user, such as around a corner or behind another 3D body. However, the present disclosure is not limited thereto.

In some embodiments, the client device includes an audio circuitry, a speaker, a microphone, or a combination thereof provide an audio interface between a user and the client device 300. The audio circuitry receives audio data from the peripherals interface, converts the audio data to an electrical signal, and transmits the electrical signal to the speaker. The speaker converts the electrical signal to human-audible sound waves. The audio circuitry converts the electrical signal to audio data and transmits the audio data to the peripherals interface for processing. The audio data is, optionally, retrieved from and/or transmitted to the memory and/or the RF circuitry by the peripherals interface. In some embodiments, the speaker is utilized to communicate one or more audible instructions associated with implementing the corresponding intervention treatment regimen for a respective subject, such as a corresponding set of instructions for the respective subject to interact with reality by way of the respective subject moving relative to the client device 300 in a first direction (e.g., away from a region of interest).

Memory 392 includes high-speed random access memory, such as DRAM, SRAM, DDR RAM, or other random access solid state memory devices, and optionally also includes non-volatile memory, such as one or more magnetic disk storage devices, optical disk storage devices, flash memory devices, or other non-volatile solid state storage devices. Memory 392 may optionally include one or more storage devices remotely located from the CPU(s) 402. Memory 392, or alternatively the non-volatile memory device(s) within memory 392, includes a non-transitory computer readable storage medium. Access to memory 392 by other components of the client device 300, such as the CPU(s) 402 and the I/O subsystem 466, is, optionally, controlled by a controller. In some embodiments, memory 392 can include mass storage that is remotely located with respect to the CPU 402. In other words, some data stored in memory 392 may in fact be hosted on devices that are external to the client device 300, but that can be electronically accessed by the client device 300 over an Internet, intranet, or other form of network 106 or electronic cable using communication interface 304.

In some embodiments, the memory 392 of the client device 300 stores:
an operating system 316 that includes procedures for handling various basic system services;
an electronic address 318 associated with the imaging device 300 that identifies the imaging device 300 within a distributed system 100;
a client application 320 for generating content for display through a graphical user interface presented on the display 308 of the imaging device 300; and
an engine 322 that allows a client application 320 to operate in conjunction with the imaging device 300.

The client device 300 includes an operating system 302 that includes procedures for handling various basic system services. The operating system 302 (e.g., iOS, DARWIN, RTXC, LINUX, UNIX, OS X, WINDOWS, or an embedded operating system such as VxWorks) includes various software components and/or drivers for controlling and managing general system tasks (e.g., memory management, storage device control, power management, etc.) and facilitates communication between various hardware and software components.

An electronic address 304 is associated with the client device 300, which is utilized to at least uniquely identify the client device 300 from other devices and components of the distributed system 100 (e.g., identify a first client device 300-1 from a second client device 300-2). In some embodiments, the electronic address 304 associated with the client device 300 is used to determine a source of a response plurality of responses provided by a respective subject for a questionnaire 208 provided by the client device 300 (e.g., receiving a respective questionnaire 208 from the resilience computer system 200 and communicating one or more responses based on the respective questionnaire 208 using the communication network 106).

In some embodiments, a client application 306 is a group of instructions that, when executed by a processor 372, generates content for presentation to the respective subject, such as a digital media including video content (e.g., video digital media), a respective questionnaire 208, a respective graphical chart, and the like. The client application 306 may generate content in response to inputs received from the user through the client device 300, such as the inputs 310 of the client device 330. As a non-limiting exemplary embodiment, in some embodiments, the client application 306 presents a resilience platform of the resilience computer system 200. Moreover, in some embodiments, the client application 306 provides a respective questionnaire 208 to a respective subject.

Each of the above identified modules and applications correspond to a set of executable instructions for performing one or more functions described above and the methods described in the present disclosure. These modules (e.g., sets of instructions) need not be implemented as separate software programs, procedures or modules, and thus various subsets of these modules are, optionally, combined or otherwise re-arranged in various embodiments of the present disclosure. In some embodiments, the memory 292 optionally stores a subset of the modules and data structures identified above. Furthermore, in some embodiments, the memory 292 stores additional modules and data structures not described above.

It should be appreciated that the client device 300 is only one example of a multifunction device that may be used by users, and that the client device 300 optionally has more or fewer components than shown, optionally combines two or more components, or optionally has a different configuration or arrangement of the components. The various components shown in FIG. 3 are implemented in hardware, software, firmware, or a combination thereof, including one or more signal processing and/or application specific integrated circuits.

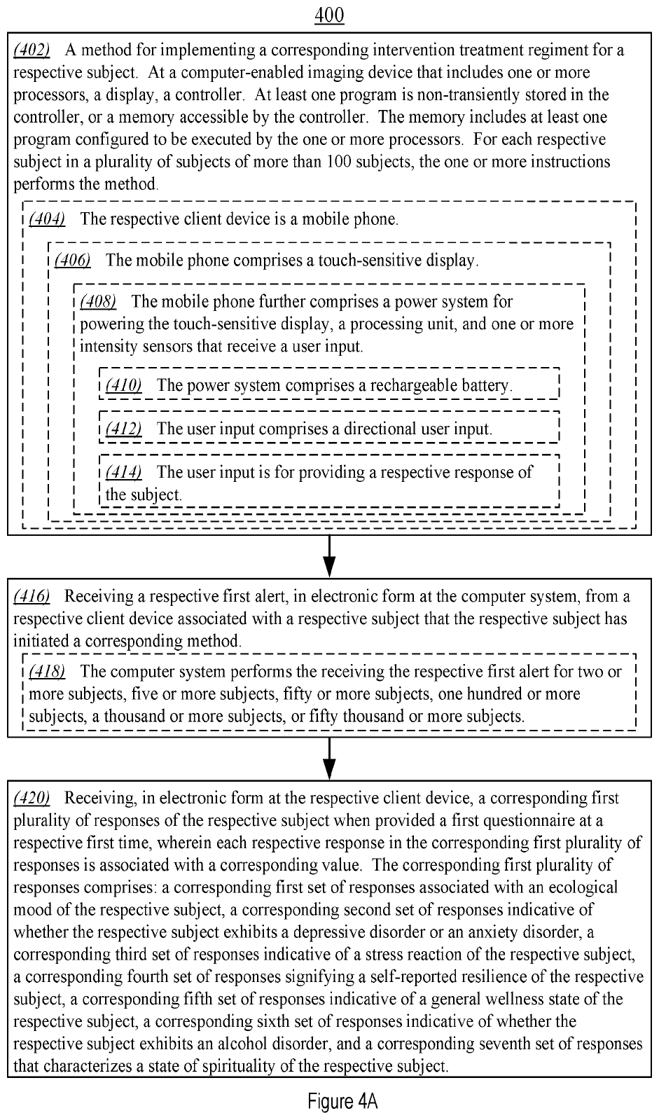

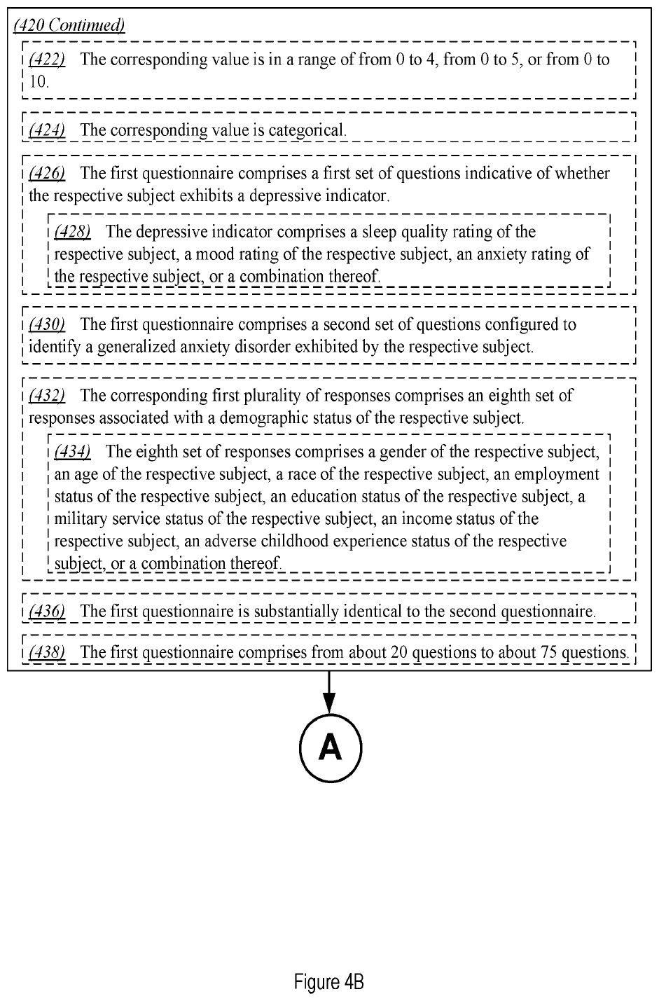

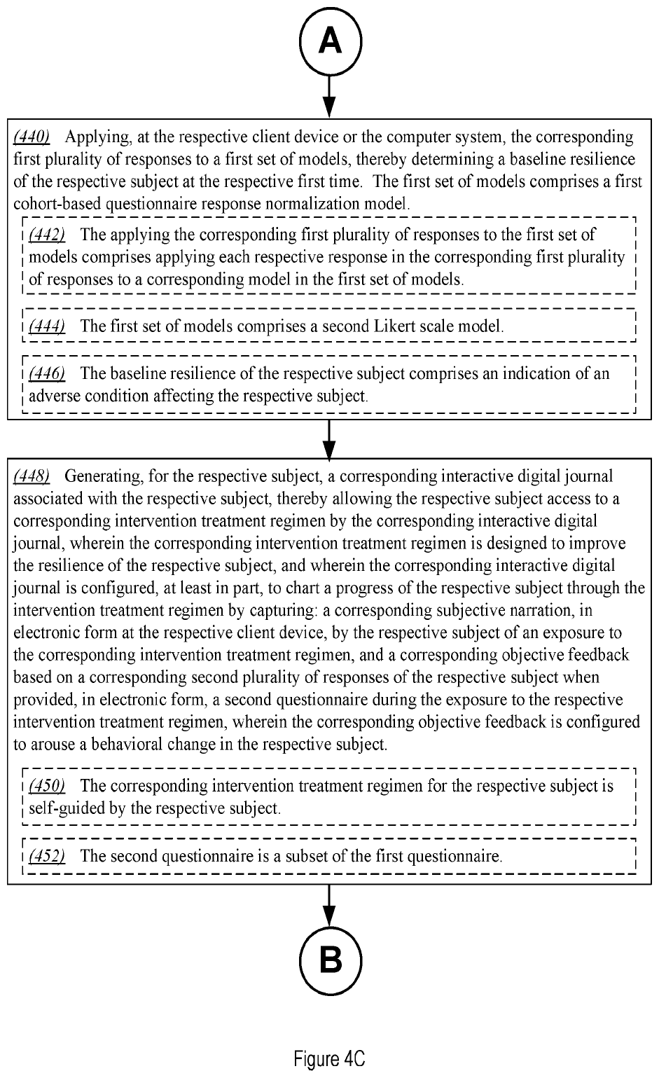

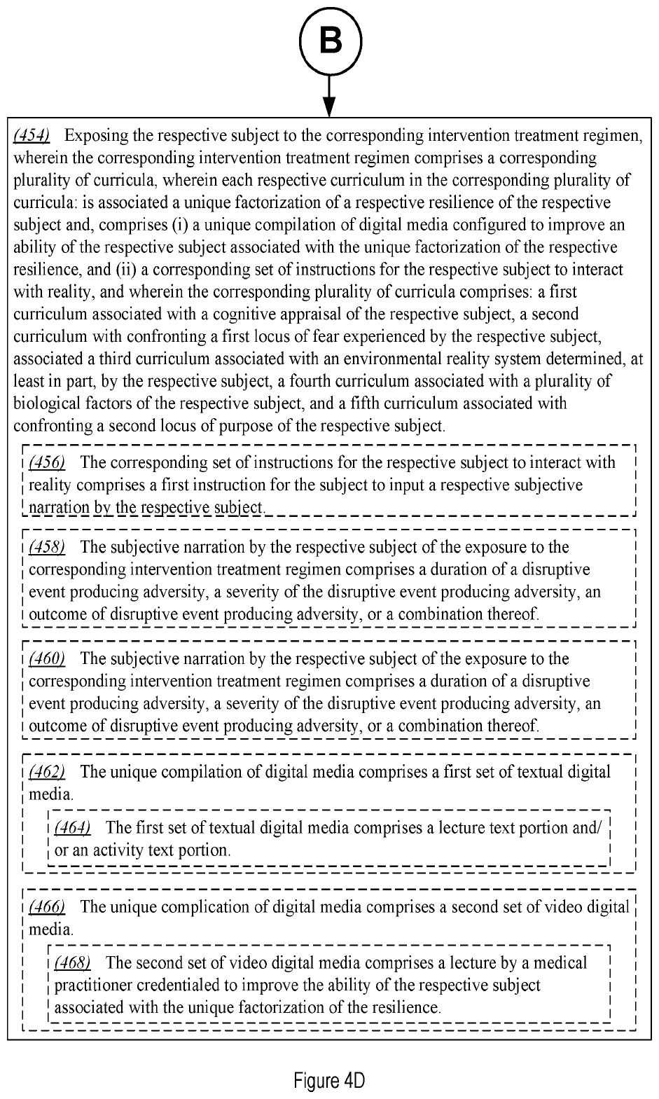

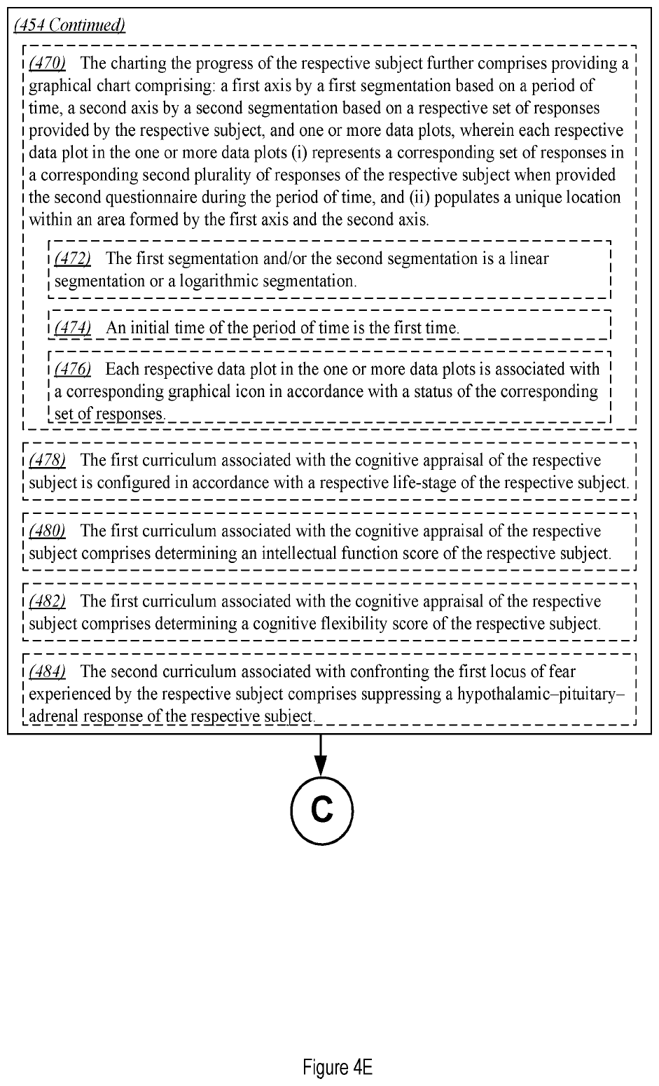

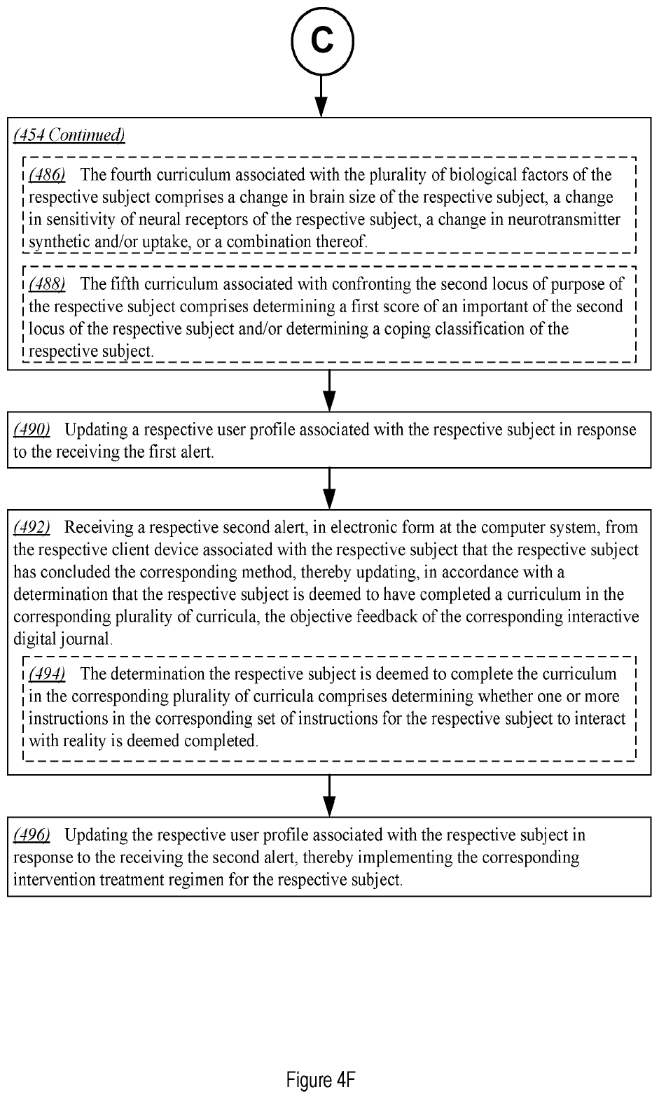

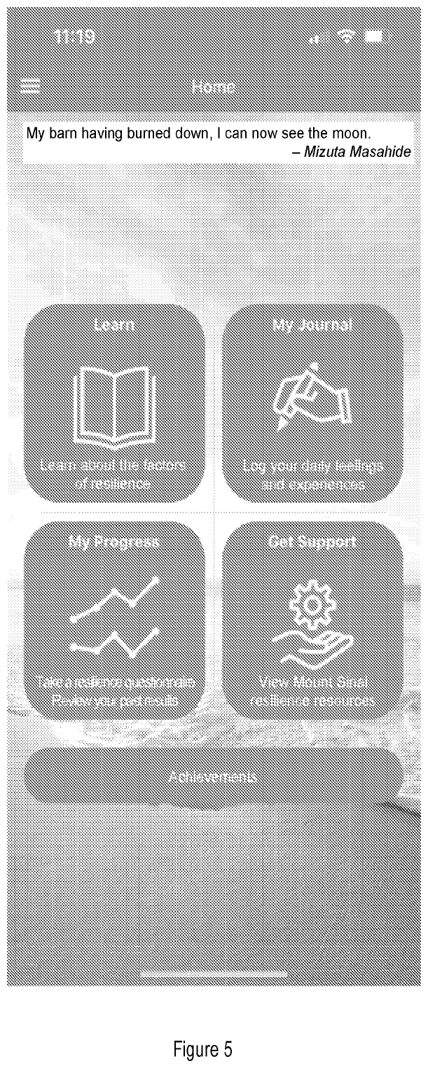

Now that a general topology of the system 100 has been described in accordance with various embodiments of the present disclosures, details regarding some processes in accordance with FIGS. 4A through 6 will be described.

Specifically, FIGS. 4A though 4F collectively illustrates a flow chart of methods (e.g., method 400) in accordance with an embodiment of the present disclosure. In the flow chart, the preferred parts of the methods 400 are shown in solid line boxes whereas optional variants of the methods, or optional equipment used by the methods, are shown in dashed line boxes.

Various modules in the memory 292 of the resilience computer system 200 and/or the client device 300 perform certain processes of the methods 400 described in FIGS. 4A though 4F, unless expressly stated otherwise. Furthermore, it will be appreciated that, in some embodiments, the processes in FIGS. 4A though 4F are encoded in a single module or any combination of modules.

Block 402. Referring to block 4002 of FIG. 4A, a method 400 for implementing the corresponding intervention treatment regimen for the respective subject.

The method 400 is performed at a computer system (e.g., resilience computer system 200 of FIGS. 2A and 2B, client device 300 of FIG. 3, etc.). The computer system 200 includes one or more processors (e.g., CPU 272) and a memory coupled to at least one processing unit of the one or more processing units, the memory storing at least one program for execution by the at least one processing unit, the at least one program including one or more instructions.

In some embodiments, for each respective subject in a plurality of subjects, the one or more instructions perform the method 400. In some embodiments, the plurality of subjects includes more than 10 subjects, more than 50 subjects, more than 100 subjects, more than 500 subjects, more than 1,000 subjects, more than 10,000 subjects, more than 50,000 subjects, more than 100,000 subjects, or the like. As such, the method 400 of the present disclosure cannot be mentally performed.

Accordingly, in some such embodiments, by implementing the corresponding intervention treatment regimen for the respective subject, the method 400 is designed as a tool for general mental health maintenance and resilience management and connects users to the mental health services available through the Center for Stress, Resilience, and Personal Growth (CSRPG) and Mount Sinai at large. See DePierro et al., 2020, “Mount Sinai's Center for Stress, Resilience and Personal Growth as a Model for Responding to the Impact of COVID-19 on Health Care Workers,” Psychiatry Res., 293, pg. 113426, which is hereby incorporated by reference in its entirety.

Block 404. Referring to block 404, in some embodiments, the respective client device is a mobile phone, such as a smart phone. However, the present disclosure is not limited thereto. For instance, in some embodiments, the respective client device 400 is a tablet or a wearable device, such as a smart watch or other type of computing garment.

Block 406. Referring to block 406, in some embodiments, the mobile phone includes a touch-sensitive display.

Block 408. Referring to block 408, in some embodiments, the mobile phone further includes a power system for powering the touch-sensitive display, a processing unit, and one or more intensity sensors that receive a user input.

Block 410. Referring to block 410, in some embodiments, the power system includes a rechargeable battery. In some embodiments, the rechargeable battery is disposed within a housing of the client device 300. As a non-limiting example, in some embodiments, the rechargeable battery includes a lithium-polymer material. In some embodiments, the rechargeable battery has a capacity of about 1000 milliamperes hours (mAh), about 1,500 mAH (e.g., 1625 mAh), about 2,000 mAh (e.g., about 1960 mAh), about 3,000 mAh, and the like. However, the present disclosure is not limited thereto.

Block 412. Referring to block 412, in some embodiments, the user input includes a directional user input, such as a uni-directional user input, a bi-directional user input, or an omni-directional user input.

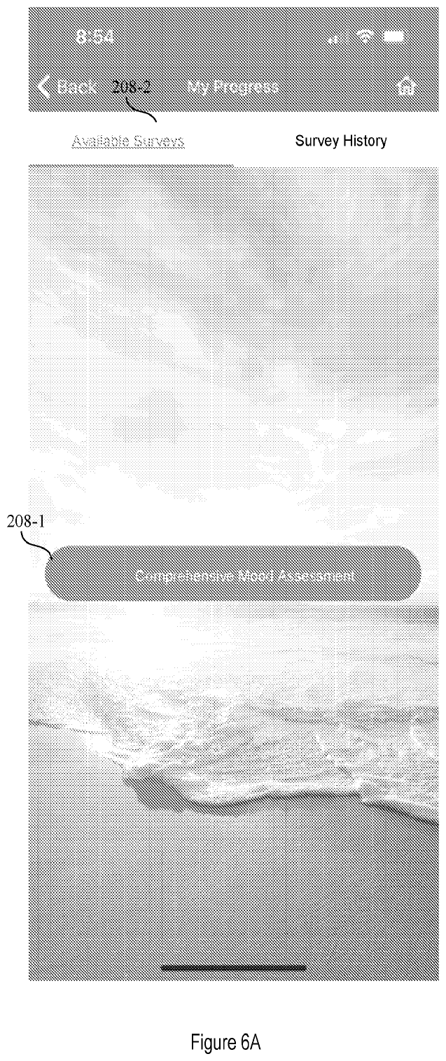

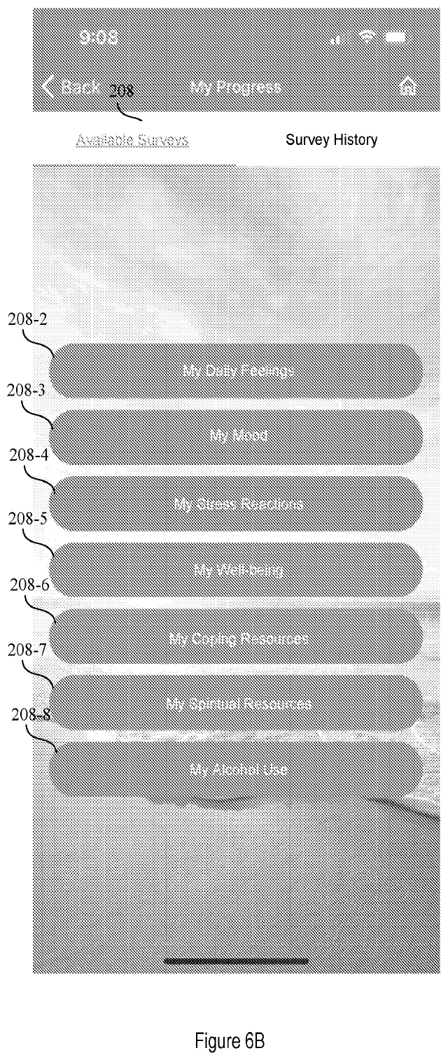

Block 414. Referring to block 414, in some embodiments, the user input is for providing a respective response of the respective subject to a corresponding question 210 of the first questionnaire 208-1. For instance, referring briefly to FIG. 6B, a set of two questions 210 from a respective questionnaire 208 presented to the respective subject is displayed through a user interface of the display 378 of the respective client device 300. Here, the respective subject has a first user input for providing a respective response of “More than half the days” responsive to a first question 210-1 of the respective questionnaire 208 that asks whether the respective subject has trouble concentrating on things, such as reading the newspaper or watching television.

Block 416. Referring to block 416, the method 400 includes receiving a respective first alert, in electronic form at the computer system, from a respective client device associated with a respective subject that the respective subject has initiated a corresponding method. For instance, referring briefly to FIG. 6A, in some embodiments, the respective subject has initiated the corresponding method when the respective subject provides a user input on an affordance region associated with a “Get Started” graphical icon. However, the present disclosure is not limited thereto.

Block 418. Referring to block 418, in some embodiments, the computer system 200 performs the receiving the respective first alert for two or more subjects, five or more subjects, fifty or more subjects, one hundred or more subjects, a thousand or more subjects, or fifty thousand or more subjects. In some such embodiments, the computer system 200 performs the receiving the respective first alert for each subject in the plurality of subjects.

Block 420. Referring to block 420, the method 400 includes receiving (e.g., in electronic form at the respective client device 300) a corresponding first plurality of responses of the respective subject when provided a first questionnaire (e.g., first questionnaire 208-1 of FIG. 2A) that includes a respective set of questions 210. Accordingly, in some such embodiments, a first time when the first questionnaire 208-1 is provided to the respective subject is determined by the computer system 200. By determining the first time the first questionnaire 208-1 is provided to the respective subject, the computer system 200 provides objective feedback 214 that is temporally relevant for the respective user when implementing the corresponding intervention treatment regimen. Furthermore, the first questionnaire 208-1 allows the computer system 200 to create a plurality of normative baseline scores (e.g., baseline resilience of block 440 of FIG. 4C) for a large (e.g., more than 100 subjects, more than 10,000 subjects, etc.), heterogeneous sample of subjects. Furthermore, the first questionnaire 208-1 allows the computer system 208-1 to develop a psychometrically-sound abbreviated measure of resilience from a broad bank of possible questions 210.

Furthermore, each respective response in the corresponding first plurality of responses is associated with a corresponding value. Accordingly, in such embodiments, by utilizing the corresponding value associated with each respective response, the method 400 is provided a quantitative mechanism to determine emotional and cognitive processes associated with the resilience of the respective subject. For instance, in some embodiments, by associating each respective response with the corresponding value, the method 200 removes uncorrelated or unnecessary features from a respective response in order to minimize a dimensionality and/or reduce an entry of given data set produced by the corresponding first plurality of responses. Further, by associating each respective response with the corresponding value, the method 200 improves the accuracy of providing object feedback 214 based on the respective response. Furthermore, by associating each respective response with the corresponding value, the method 200 decreases an elapsed period of time that is required to process the corresponding first plurality of responses. For instance, in some embodiments, the corresponding first plurality of responses is mapped to an index based on the corresponding value associated with each respective response in the corresponding first plurality of responses.

In some embodiments, the first questionnaire 208-1 includes (e.g., subsumes some or all of) a first set of questions from a Revised Life Orientation questionnaire 208 (LOT-R questionnaire), a second set of questions from a International Positive and Negative Affect Schedule-Short Form questionnaire 208 (I-PANAS-SF questionnaire 208), a third set of questions from a Modified Medical Outcomes Study Social Support Scale-5, including the Provision of Support Scale, questionnaire (MOS-5 questionnaire), a fourth set of questions form a 15 question from the Brief-COPE questionnaire; a fifth set of questions from a Godin Leisure-Time Exercise questionnaire; a sixth set of questions from a Duke University Religion Index questionnaire (DUREL questionnaire), a seventh set of questions from a Purpose in Life Test-Short Form questionnaire (PIL-SF questionnaire), an eighth set of questions (e.g., question number 6) from a Pittsburgh Sleep Quality Index questionnaire (PSQI questionnaire), a ninth set of questions (e.g., question number 4) from a Insomnia Severity Index questionnaire, a tenth set of questions from a Patient Health Questionnaire (PHQ-8 questionnaire), an eleventh set of questions from a Generalized Anxiety Disorder 2-question questionnaire (GAD-2 questionnaire), a twelveth set of questions from a 4-question version of a PTSD Checklist for a DSM-5 questionnaire (PCL-5 questionnaire), a thirteenth set of questions from a Life Events Checklist for the DSM-5 questionnaire (LEC-5 questionnaire), a fourteenth set of questions from an Acceptance and Action Questionnaire (AAQ-2 questionnaire), a fifteenth set of questions from a Posttraumatic Growth Inventory-Short Form questionnaire (PTGI-SF questionnaire), a sixteenth set of questions from a 10-question Connor-Davidson Resilience Scale questionnaire (CD-RISC-10 questionnaire), a seventeenth set of questions (e.g., question number 3) from a Mini-Z questionnaire, an eightieth set of questions (e.g., question number nine 210-9) from a Curiosity and Exploration Inventory—II questionnaire, a nineteenth set of questions 210 from a three-question Loneliness scale questionnaire, or a combination thereof. Additional details and information regarding the first questionnaire 208-1 can be found at Scheier et al., 1994, “Distinguishing optimism from neuroticism (and trait anxiety, self-mastery, and self-esteem): A re-evaluation of the Life Orientation Test,” Journal of Personality and Social Psychology, 67, pg. 1063-1078; Thompson, E., 2007, “Development and validation of an internationally reliable short-form of the Positive and Negative Affect Schedule (PANAS),” Journal of Cross-Cultural Psychology, 38(2), pg. 227; Sherbourne et al., 1991, “The MOS social support survey,” Social Science & Medicine, 32(6), pg. 705; Carver, C., 1997, “You want to measure coping but your protocol is too long: Consider the brief cope,” International Journal of Behavioral Medicine, 4(1), pg. 92; Godin, G., 2011, “The Godin-Shephard Leisure-Time Activity Questionnaire,” Health & Fitness Journal of Canada, 4(1), pg. 18; Koenig et al., 2010, “The Duke University Religion Index (DUREL): A five-item measure for use in epidemiological studies,” Religions, 1, pg. 78; Schulenberg et al., 2011, “The Purpose in Life Test-Short Form: Development and psychometric support,” Journal of Happiness Studies, 12, pg. 861; Buysse et al., 1989, “The Pittsburgh Sleep Quality Index (PSQI): A new instrument for psychiatric research and practice,” Psychiatry Research, 28(2), pg. 193; Morin, C., 1993, “Insomnia: Psychological assessment and management,” The Guilford Press, print; Kroenke, et al., 2009, “The PHQ-8 as a measure of current depression in the general population,” J Affect Disord., 114(1-3), pg. 163; Kroenke et al., 2007, Anxiety disorders in primary care: Prevalence, impairment, comorbidity, and detection,” Ann Intern med, 146, pg. 317; Geier et al., 2020, “Validation of abbreviated four- and eight-item versions of the PTSD Checklist for DSM-5 in a traumatically injured sample,” Journal of Traumatic Stress, 33, pg. 218; Weathers et al., 2013, “The Life Events Checklist for DSM-5 (LEC-5)” National Center for PTSD, available at ptsd.va.gov (access Jan. 22, 2022); Bond et al., 2011, “Preliminary psychometric properties of the Acceptance and Action Questionnaire—II: A revised measure of psychological inflexibility and experiential avoidance,” Behavior Therapy, 42, pg. 676; Campbell-Sills et al., 2007, “Psychometric analysis and refinement of the Connor-Davidson Resilience Scale (CD-RISC): Validation of a 10-item measure of resilience,” Journal of Traumatic Stress, 20(6), pg. 1019; Cann et al., 2010, “A short form of the Posttraumatic Growth Inventory” Anxiety, Stress & Coping, 23(2), pg. 127; Kashdan et al., 2009, “The curiosity and exploration inventory-II: Development, factor structure, and psychometrics,” Journal of research in personality, 43(6), pg. 987; Linzer et al., 2000, “Managed care, time pressure, and physician job satisfaction: Results from the Physician Worklife Study,” J. Gen. Intern. Med., 15, pg. 441, each of which is hereby incorporated by reference in its entirety.

In some embodiments, each set of questions 210 in the first questionnaire 208-1 includes at least one question 210, at least 2 questions, at least 3 questions, at least 4 questions, at least 5 questions, at least six questions, or at least ten questions.

Block 422. Referring to block 422 of FIG. 4B, in some embodiments, the corresponding value is in a range of from 0 to 4, from 0 to 5, or from 0 to 10.

In some embodiments, the range of from 0 to 4 corresponds to a plurality of responses by the respective subject from: 0 is equivalent to a first response of not at all true, 1 is equivalent to a second response of rarely true, 2 is equivalent to a third response of sometimes true, 3 is equivalent to a fourth response of often true, and 4 is equivalent to a fifth response of true nearly all of the time. However, the present disclosure is not limited thereto. For instance, the range of from 0 to 4 corresponds to the plurality of responses by the respective subject from: 0 is equivalent to the first response of not at all, 1 is equivalent to the second response of a little bit, 2 is equivalent to the third response of somewhat, 3 is equivalent to a fourth response of moderately, and 4 is equivalent to a fifth response of true nearly very. Accordingly, the present disclosure allows for the conversion of complex responses provided by the respective subject into the corresponding value, which allows for the responses to be applied to one or more models 218 in a set of models 218.

As a non-limiting example, in some embodiments, each respective question asks the respective subject to rate how well a corresponding question applies the respective subject when considering a period of time (e.g., a day, a week, two weeks, a month, a year, a lifetime, etc.). For instance, in some embodiments, each response to each of the questions in the respective set of questions is converted to a value in a range from 5 (e.g., associated with a response of all of the time) to 0 (e.g., associated with a response of none of the time).

In some embodiments, a total of the corresponding values of each response has a theoretical range from 0 (e.g., responses of absence of well-being) to an positive integer (e.g., 25) (e.g., responses of maximal well-being). However, the present disclosure is not limited thereto.

As another non-limiting example, in some embodiments, a first set of question associated with a PHQ-8 questionnaire 208 and a second set of questions associated with a GAD-2 questionnaire are presented on a scale with a range of corresponding values from 0 to about 3, with higher scores indicating more severe depression and anxiety, respectively, exhibited by the respective subject. In some embodiments, the first set of questions associated with the PHQ-8 questionnaire has the range of the corresponding value from 10 or higher to indicate probable depression exhibited by the respective subject. In some embodiments, the range of the corresponding value of 3 or higher on the GAD-2 by the respective subject indicates clinically significant anxiety problems.

As another non-limiting example, in some embodiments, the third set of questions associated with a CD-RISC2 questionnaire 208 has a range of the corresponding value from 0 (e.g., not true at all) to 4 (e.g., true nearly all the time). In some such embodiments, higher scores (e.g., large value numbers) indicate greater resilience exhibited by the respective subject. In some embodiments, based on a normative data (e.g., as determined by a first cohort-based questionnaire response normalization model 218-1), a corresponding value of 5 or lower is used to indicate current problems with resilience exhibited by the respective subject (e.g., for purposes of providing objective feedback for the respective subject). See Vaishnavi et al., 2007.

As yet another non-limiting example, in some embodiments, a fourth set of questions associated with a World Health Organization 5 question 210 questionnaire 210 (e.g., WHO-5 questionnaire 208) are presented with a range of the corresponding value from 5 (e.g., all the time) to 0 (e.g., at no time scale). In some embodiments, higher values indicate greater well-being exhibited by the respective subject. In some embodiments, the corresponding values determined by each respective question in the fourth set of questions factored (e.g., summed and then multiplied by four) to yield an index (e.g., 0-100 index). In some such embodiments, the corresponding value at or below 50 is suggestive of depression exhibited by the respective subject. However, the present disclosure is not limited thereto. See Topp et al., 2015, “The WHO-5 Well-Being Index: A Systematic Review of the Literature,” Psychother. Psychosom., 84(3), pg. 167, which is hereby incorporated by reference in its entirety.

Block 424. Referring to block 424, in some embodiments, the corresponding value is categorical. For instance, in some such embodiments, the corresponding value is a categorical value that has two or more categories lacking intrinsic ordering to the two or more categories, five or more categories, ten or more categories, or the like. As a non-limiting example, in some embodiments, a first categorical class of a corresponding value is associated with a status of the respective subject, a citizenship of the respective subject, a religion of the respective subject, and the like.

In some embodiments, the corresponding first plurality of responses includes a corresponding first set of responses associated with an ecological mood of the respective subject. As described supra, in some embodiments, the ecological mood of the respective subject includes how the respective subject feels on a quantitative scale about a relationship with an environment of the respective subject.

In some embodiments, the corresponding first plurality of responses includes a corresponding second set of responses indicative of whether the respective subject exhibits a depressive disorder or an anxiety disorder. In some embodiments, the depressive disorder is a depression (e.g., depressive mood, reduced serotonin, etc.) exhibited by the respective subject that is related to the normal emotions of sadness and bereavement, but the depression does not remit when an external cause of the depression to dissipate, and the depression is disproportionate to their cause. Moreover, in some embodiments, the anxiety disorder is a separation anxiety disorder, a selective mutism, a specific phobia, a social anxiety disorder, a panic disorder, an agoraphobia, a generalized anxiety disorder, a substance-induced anxiety disorder, or an anxiety disorder due to a medical condition of the respective subject. Additional details and information regarding depressive disorders and anxiety disorders is found in Kennis et al., 2020, “Prospective Biomarkers of Major Depressive Disorder: A Systematic Review and Meta-analysis,” Molecular Psychiatry 25, pp. 321-338; and Zuckerman et al., 2018, “Recognition and Treatment of Cognitive Dysfunction in Major Depressive Disorder,” Frontiers in Psychiatry 9, pg. 655, each of which is hereby incorporated by reference in its entirety. In some embodiments, any of the questions on the Posttraumatic Stress Disorder Checklist-5 (PCL-5) (See, Blevins et al., 2015, “The posttraumatic stress disorder checklist for DSM-5 (PCL-5): Development and initial psychometric evaluation”, J. Trauma Stress 28(6), pp. 489-498, which is hereby incorporated by reference), Patient Health Questionnaire-2 (PHQ-2) (See, Kroenke et al., 2009, “An ultra-brief screening scale for anxiety and depression: the PHQ-4,” Psychosomatics 50(6), pp. 613-621, which is hereby incorporated by reference), or Generalized anxiety disorder-2 (GAD-2) (Id., which is hereby incorporated by reference) are included in the first questionnaire to identify and/or quantify any depressive disorder or anxiety disorder exhibited by the subject.

In some embodiments, the corresponding first plurality of responses includes a corresponding third set of responses indicative of a stress reaction of the respective subject. In some embodiments, the stress reaction of the respective subject is one or more responses (e.g., digital responses or biological responses) by the respective subject that is elicited by one or more corresponding stressors. For instance, in some embodiments, the stress reaction of the respective subject in the one or more responses includes an increase in circulating levels of catecholamines and glucocorticoids, which promote increases in cardiovascular tone. In some embodiments, the stress reaction of the respective subject includes a change in attentional process, such as diminished episodic memory capacity during a period of stress. Additional details and information regarding the stress reaction of the respective subject that can be monitored through the third set of responses is found in Arnsten, 2009, “Stress signaling pathways that impair prefrontal cortex structure and function, Nature 10(6), pp. 410-422; and Meaney, 2001, “Maternal care, gene expression, and the transmission of individual differences in stress reactivity across generations,” Annual Review of Neuroscience 24(1), pg. 1161, which is hereby incorporated by reference in its entirety.

In some embodiments, the corresponding first plurality of responses includes a corresponding fourth set of responses signifying a self-reported resilience of the respective subject. For instance, in some embodiments, the respective subject is asked to opine regarding how the respective subject views themselves internally as being able to handle adversity and/or disruptive events. However, the present disclosure is not limited thereto.

In some embodiments, the corresponding first plurality of responses includes a corresponding fifth set of responses indicative of a general wellness state of the respective subject. For instance, in some embodiments, the general wellness state of the respective subject is a comparison of one or more responses provided by the respective subject with one or more corresponding mean values from a general population cohort (e.g., to be used as a criterion of remission by the respective subject) or with one or more mean values from other clinical populations irrespective of the disease entity or condition under examination. In some embodiments, the corresponding fifth set of responses indicative of the general wellness state of the respective subject is responsive to a respective set of questions (e.g., a set of questions in the first questionnaire 208-1, a second questionnaire 208-2, etc.) including: a first question of “I have felt cheerful and in good spirits”; a second question of “I have felt calm and relaxed,” a third question of “I have felt active and vigorous,” a fourth question of “I woke up feeling fresh and rested,” a fifth question of “My daily life has been filled with things that interest me,” or a combination thereof.

In some embodiments, the corresponding first plurality of responses includes a corresponding sixth set of responses indicative of whether the respective subject exhibits an alcohol disorder. For instance, in some embodiments, the corresponding sixth set of responses indicate the respective subject exhibits an alcohol disorder based on an intensity or frequency of drinking by the respective subject, such as a total amount of alcohol consumed within a certain assessment period of time (e.g., four or more alcoholic servings per week, five or more alcoholic serving per week, six or more alcoholic serving per week, seven or more alcoholic serving per week, etc.). Additional details and information regarding alcohol disorders are found in Miller et al., 2001, “How effective is alcoholism treatment in the United States?,” Journal of studies on alcohol, 62(2), pg. 211; Bush et al., 1998, the AUDIT Alcohol Consumption Questions (AUDIT-C): An Effective Brief Screening Test for Problem Drinking,” Archives of Internal Medicine, 158(16), pg. 1789, each of which is hereby incorporated by reference in its entirety.

In some embodiments, the corresponding first plurality of responses includes a corresponding seventh set of responses that characterizes a state of spirituality of the respective subject. Furthermore, in some embodiments, the first plurality of responses includes a respective set of responses associated with: an optimism of the respective subject; an ability to facing fears of the respective subject (e.g., a first locus of fear experienced by the respective subject); a circle of social support of the respective subject (e.g., an environmental reality system determined, at least in part, by the respective subject) that includes one or more friends, one or more family members, one or more peers (e.g., another subject) associated with the respective friend; a spirituality of the respective subject; a physical health of the respective subject (e.g., a plurality of biological factors of the respective subject); a meaning and growth of the respective subject over a period of time (e.g., a mental maturity of the respective growth over the period of time); a value of altruism held by the respective subject (e.g., an ecological mood of the respective subject); a cognitive flexibility of the respective subject; an emotional flexibility of the respective subject; a moral compass of the respective subject; a presence of one or more role models in the life of the respective subject; a bran fitness value of the respective subject, or a combination thereof.

For instance, in some embodiments, the set of responses associated with: an optimism of the respective subject is responsive to a corresponding set of questions 210 of the first questionnaire 208-1 including: a first question of “I have made efforts to remain hopeful or positive about the future,” a second question of I have done things to make my daily life better, even in small ways,” a third question of “I told myself that the current challenges I face are temporary,” and one or more questions associated with an efficacy rating of each of the aforementioned questions (e.g., an efficacy rating of the first question, an efficacy rating of the second question, etc.).

In some embodiments, the set of responses associated with an ability to facing fears of the respective subject (e.g., a first locus of fear experienced by the respective subject) is responsive to a corresponding set of questions 210 of the first questionnaire 208-1 including: a first question of “I have tended to confront my fears and problems head on,” a second question of “I have learned something important about myself by facing my fears,” a third question of “I have tended to plan, prepare, and train for situations I expect to be stressful or frightening,” and one or more questions associated with an efficacy rating of each of the aforementioned questions (e.g., an efficacy rating of the first question, an efficacy rating of the second question, etc.).

In some embodiments, the set of responses associated with a circle of social support of the respective subject (e.g., an environmental reality system determined, at least in part, by the respective subject) that includes one or more friends, one or more family members, one or more peers (e.g., another subject) associated with the respective friend are responsive to a corresponding set of questions 210 of the first questionnaire 208-1 including: a first question of “I have sought the support of others,” a second question of “I have invested time in giving support to others,” a third question of “I have reminded myself that I am not alone,” and one or more questions associated with an efficacy rating of each of the aforementioned questions (e.g., an efficacy rating of the first question, an efficacy rating of the second question, etc.).

In some embodiments, the set of responses associated with a spirituality of the respective subject is responsive to a corresponding set of questions 210 of the first questionnaire 208-1 including: a first question of “I have engaged in spiritual practices like prayer, meditation, or participating in a faith gathering in person or virtually,” a second question of “I believed that my life has a purpose,” a third question of “I have felt a meaningful connection to something greater than myself,” and one or more questions associated with an efficacy rating of each of the aforementioned questions (e.g., an efficacy rating of the first question, an efficacy rating of the second question, etc.).

In some embodiments, the set of responses associated with a physical health of the respective subject (e.g., a plurality of biological factors of the respective subject) is responsive to a corresponding set of questions 210 of the first questionnaire 208-1 including: a first question of “I have done my best to get enough sleep,” a second question of “I have made sure to eat nutritious food(s) each day, whenever I have had access to it,” a third question of “I have pushed myself to physically exercise,” and one or more questions associated with an efficacy rating of each of the aforementioned questions (e.g., an efficacy rating of the first question, an efficacy rating of the second question, etc.).

In some embodiments, the set of responses associated with a meaning and growth of the respective subject over a period of time (e.g., a mental maturity of the respective growth over the period of time) is responsive to a corresponding set of questions 210 of the first questionnaire 208-1 including: a first question of “I believe I have grown in a positive way from challenges in my life,” a second question of “I believed that having a meaningful life is possible, even if very difficult times,” a third question of “I have engaged in activities that bring me a sense of meaning and purpose,” and one or more questions associated with an efficacy rating of each of the aforementioned questions (e.g., an efficacy rating of the first question, an efficacy rating of the second question, etc.).

In some embodiments, the set of responses associated with a value of altruism held by the respective subject (e.g., an ecological mood of the respective subject) is responsive to a corresponding set of questions 210 of the first questionnaire 208-1 including: a first question of “I have engaged in service or volunteerism to help others,” a second question of “I have provided emotional, financial, or another kind of tangible support to other people going through difficulties,” a third question of “I believed that my challenging life experiences have made me more able to help others now or in the future,” and one or more questions associated with an efficacy rating of each of the aforementioned questions (e.g., an efficacy rating of the first question, an efficacy rating of the second question, etc.).

In some embodiments, the set of responses associated with a cognitive flexibility of the respective subject is responsive to a corresponding set of questions 210 of the first questionnaire 208-1 including: a first question of “I have tried find something good in stressful situations,” a second question of “I have actively tried to change or challenge overly negative or critical thoughts about myself or others,” a third question of “I have accepted difficult emotions, situations, or people that I cannot change right now,” and one or more questions associated with an efficacy rating of each of the aforementioned questions (e.g., an efficacy rating of the first question, an efficacy rating of the second question, etc.).

In some embodiments, the set of responses associated with an emotional flexibility of the respective subject is responsive to a corresponding set of questions 210 of the first questionnaire 208-1 including: a first question of “I have talked about my feelings with others,” a second question of “I have devoted time to noticing and understanding my emotional and bodily reactions to stressful situations,” a third question of “I have slowed myself down ‘in the moment’ to manage intensely negative emotional reactions,” and one or more questions associated with an efficacy rating of each of the aforementioned questions (e.g., an efficacy rating of the first question, an efficacy rating of the second question, etc.).

In some embodiments, the set of responses associated with a moral compass of the respective subject is responsive to a corresponding set of questions 210 of the first questionnaire 208-1 including: a first question of “I have had a clear sense of personal values—what's truly important to me in my life,” a second question of “I have done what I thought or felt was right in my day-to-day life,” a third question of “I have worked to forgive myself or others for doing something that disagreed with my values,” and one or more questions associated with an efficacy rating of each of the aforementioned questions (e.g., an efficacy rating of the first question, an efficacy rating of the second question, etc.).

In some embodiments, the set of responses associated with a presence of one or more role models in the life of the respective subject are responsive to a corresponding set of questions 210 of the first questionnaire 208-1 including: a first question of “I have used lessons learned from role models in my life,” a second question of “I have reached out for and received advice on how to manage challenges from friends, mentors, family members, spiritual leaders, or teachers,” a third question of “I have been a positive role model for others,” and one or more questions associated with an efficacy rating of each of the aforementioned questions (e.g., an efficacy rating of the first question, an efficacy rating of the second question, etc.)

In some embodiments, the set of responses associated with a bran fitness value of the respective subject is responsive to a corresponding set of questions 210 of the first questionnaire 208-1 including: a first question of “I have devoted time to my hobbies or interests,” a second question of “I have engaged in challenging activities that require skill, planning, and my full attention,” a third question of “I have engaged in challenging activities that require skill, planning, and my full attention,” and one or more questions associated with an efficacy rating of each of the aforementioned questions (e.g., an efficacy rating of the first question, an efficacy rating of the second question, etc.).

By way of example, in some embodiments, the first questionnaire 208-1 includes at least 5 sets of questions 210, at least 10 sets of questions, at least 15 sets of questions, or the like (e.g., 5 sets of questions, 6 sets of questions, 7 sets of questions, 10 sets of questions, 12 sets of questions, 15 sets of questions, 20 sets of questions, etc.). Each set of questions 210 of the first questionnaire 208-1 is configured to elicit a corresponding set of responses in the first corresponding plurality of responses provided by the subject. In some embodiments, each set of questions 210 in the first questionnaire 208-1 is itself a questionnaire 208 (e.g., the first questionnaire 208-1 is a summation of a plurality of questionnaires 208).

In some embodiments, the first questionnaire 208-1 includes a first set, or subset, of questions associated a frequency rating of an aspect of the respective subject (e.g., optimism frequency rating, facing fears frequency rating, social support frequency rating, spirituality frequency rating, physical health frequency rating, meaning and growth frequency rating, altruism frequency rating, cognitive flexibility frequency rating, emotional flexibility frequency rating, moral compass frequency rating, role models frequency rating, brain fitness frequency rating, etc.), a second set, or subset, of questions associated with the an efficacy (e.g., helpfulness) of the aspect of the respective subject (e.g., optimism efficacy rating, facing fears efficacy rating, social support efficacy rating, spirituality efficacy rating, physical health efficacy rating, meaning and growth efficacy rating, altruism efficacy rating, cognitive flexibility efficacy rating, emotional flexibility efficacy rating, moral compass efficacy rating, role models efficacy rating, brain fitness efficacy rating, etc.). By way of example, in some embodiments, the first set of questions includes one or more questions associated with a frequency rating by the subject for confronting a first locus of fear experienced by the respective subject and a second set of questions includes one or more questions associated with an efficacy rating by the subject for confronting the first locus of fear experienced by the respective subject. However, the present disclosure is not limited thereto.

Additional details and information regarding the first questionnaire 208-1 and the corresponding first plurality of responses can be found at Golden et al., 2021, “A Resilience-Building App to Support the Mental Health of Health Care Workers in the COVID-19 Era: Design Process, Distribution, and Evaluation,” JMIR Formative Research, 5(5), e26590; DePierro et al., 2021, “Developments in the First Year of a Resilience-focused Program for Health Care Workers,” Psychiatry Research, 306, pg. 114280; King et al., 2017, “Determining Best Methods to Screen for Religious/Spiritual Distress,” Supportive Care in Cancer, 25(2), pg. 471, each of which is hereby incorporated by reference in its entirety

Referring briefly to FIGS. 7A through 7C, in some embodiments, in some embodiments, the respective subject visualizes the first questionnaire 208-1 through a user interface (e.g., user interface 376 of client device 300 FIG. 3). In some embodiments, the respective subject is introduced to the corresponding intervention treatment regimen for the respective subject by providing the first questionnaire 208-1 as a baseline survey (e.g., “Comprehensive Mood Assessment” questionnaire 208 of FIG. 6A. For instance, in some embodiments, the first questionnaire 208-1 includes each question 210 of a respective questionnaire in a questionnaire library 206. Accordingly, the first questionnaire 208-1 is provided by the computer system 200 to the respective subject in order to determine validated objective feedback 214. In some embodiments, each question 210 of the first questionnaire 208-1 is presented to the respective subject in plain language, as opposed to clinical terminology, in order to reduce a cognitive burden of the respective subject and provide improved precision and accuracy when determining the objective feedback 214 best for the respective subject. However, the present disclosure is not limited thereto.

Block 464. Referring to block 426, in some embodiments, the first questionnaire 208-1 includes a first set of questions 210 (e.g., first question 210-1, second question 210-2, etc.) indicative of whether the respective subject exhibits a depressive indicator, anxiety disorder and/or indicative of the mood of the subject. As a non-limiting example, in some embodiments, the first set of questions includes an ecological mood assessment (“My Daily Feelings” questionnaire 208 of FIG. 6B). In some embodiments, the ecological mood assessment is configured to determine how the respective subject feels about one or more relationships between the respective subject and the environment of the respective subject (e.g., materials and organisms at the individual, population, community, ecosystems, and/or biosphere level). This ecological mood assessment allows the computer system 200 to evaluate variations in daily physical activity and mood of the subject against a cohort of subjects. As such, the corresponding plurality of responses of the respective subject provided responsive to the ecological mood assessment questions 210 includes obtaining real-time data about the physical activity and/or mood of the respective subject in the environment of the respective subject repeatedly over time. In some embodiments, the first set of questions 210 is presented to the respective subject as a meter (e.g., graphical chart including one axis) with a plurality of emoticons indicating a scale having a range of from about 1 to about 7 (e.g., from sadness to happiness), for the respective subject to rate their mood (e.g., past and/or instantaneous mood the respective subject when conducting the first questionnaire 208-1). Examples of the factors queried and possible questions used to this end within the first set of questions 210 is found at Kim et al., 2016, “Depression screening using daily mental-health ratings from a smartphone application for breast cancer patients,” J. Med. Internet Res., 18(8), pg. 216; and Kanning et al., “Be active and become happy: An ecological momentary assessment of physical activity and mood,” Journal of Sport and Exercise Psychology, 32(2), pg. 253, each of which is hereby incorporated by reference in its entirety.

Block 428. Referring to block 428, in some embodiments, the depressive indicator includes a sleep quality rating of the respective subject (e.g., a restoration status after sleeping, a difficulty falling asleep, a difficulty getting up after sleeping, a satisfaction with sleep, a difficulty in maintain sleep, etc.), a mood rating of the respective subject, an anxiety rating of the respective subject, or a combination thereof. Additional details and information regarding the sleep quality rating of the respective subject can be found at Ohayon et al., 2017, “National Sleep Foundation's Sleep Quality Recommendations: First Report,” Sleep Health, 3(1), pg. 6, which is hereby incorporated by reference in its entirety.

Block 430. Referring to block 430, in some embodiments, the first questionnaire includes a second set of questions configured to identify a generalized anxiety disorder exhibited by the respective subject. As a non-limiting example, in some embodiments, the second set of questions 210 includes an eight-question 210 patient health questionnaire (e.g., PHQ-8 questionnaire) and a two-question general anxiety disorder questionnaire 208 (e.g., GAD-2 questionnaire). In some embodiments, the PHQ-8 and GAD-2 questionnaires 208 screen for depression and overall anxiety, respectively, over the past two weeks. See Kroenke et al., 2009, “The PHQ-8 as a measure of current depression in the general population,” J. Affect. Disord., 114(1-3), pg. 163; Plummer et al., 2016, “Screening for anxiety disorders with the GAD-7 and GAD-2: A systematic review and diagnostic metaanalysis,” Gen. Hosp. Psychiatry, 39, pg. 24, each of which is hereby incorporated by reference in its entirety. In some embodiments, the PHQ-8 questionnaire 208 is identical to the PHQ-9 questionnaire 208, except that a respective question 210 pertaining to suicidality is omitted from the PHQ-8 questionnaire 208. See Kroenke et al., 2001, “The PHQ-9: Validity of a brief depression severity measure,” J. Gen. Intern. Med., 16(9), pg. 606, which is hereby incorporated by reference in its entirety.

In some embodiments, the first questionnaire 208-1 includes a third set of questions 210 that is associated with a Connor-Davidson Resilience Scale (CD-RISC) that includes two questions 210 (e.g., CD-RISC2) or about ten questions (e.g., CD-RISC10) (e.g., “My Copying Resources” questionnaire 208 of FIG. 6B). In some embodiments, the CD-RISC2 set of questions 210 is a validated short form of the CD-RISC. See Vaishnavi et al., 2007, “An abbreviated version of the Connor-Davidson Resilience Scale (CD-RISC), the CD-RISC2: Psychometric properties and applications in psychopharmacological trials,” Psychiatry Res., 152(2-3), pg. 293; Connor et al., 2003, “Development of a new Resilience scale: The Connor-Davidson Resilience scale (CD-RISC),” Depress. Anxiety, 18(2), pg. 76, each of which is hereby incorporated by reference in its entirety.

Block 432. Referring to block 432, in some embodiments, the corresponding first plurality of responses includes an eighth set of responses associated with a demographic status of the respective subject. In some embodiments, the eighth set of responses associated with the demographic status of the respective subject is utilized by the computer system 200 in order to improve the personalization of the objective feedback 214 that is provided to the respective subject based a demographical aspect of the respective subject. By utilizing the demographic status of the respective subject, the computer system 200 better understands the breakdown of a population of subjects (e.g., at least 5 subjects, at least 50 subjects, at least 500 subjects, at least 50,000 subjects, etc.) to better address and target outreach to an underserved population of subjects.

Block 434. Referring to block 434, in some embodiments, the eighth set of responses includes a gender of the respective subject, an age of the respective subject, a race or ethnicity of the respective subject, an employment status of the respective subject (e.g., whether or not the respective subject is currently employed or seeking employment), an education status of the respective subject (e.g., a highest level of education completed and/or attempted by the respective subject), a military service status of the respective subject (e.g., a branch of service of the respective subject and/or a retired status of the respective subject), an income status of the respective subject (e.g., a gross annual household income associated with the respective subject, a number of dependents associated with the respective subject, etc.), an adverse childhood experience status of the respective subject (e.g., whether or not the respective subject experience childhood abuse), or a combination thereof. In some embodiments, each respective response in the eight set of responses is associated with a categorical corresponding value. However, the present disclosure is not limited thereto.

Block 436. Referring to block 436, in some embodiments, first questionnaire 208-1 is substantially identical to the second questionnaire 208-1. However, the present disclosure is not limited thereto.

Block 438. Referring to block 438, in some embodiments, the first questionnaire 208-1 includes from about 20 questions 210 to about 75 questions 210 (e.g., 42 questions 210). However, the present disclosure is not limited thereto. For instance, in some embodiments, the first questionnaire 208-1 includes about 10 questions, about 20 questions (e.g., 19 questions) about 27 questions (e.g., 30 questions 210), about 90 questions, or the like.

Block 440. Referring to block 440 of FIG. 4C, the method 400 includes applying (e.g., at the respective client device 300 and/or the computer system 200), the corresponding first plurality of responses to a first set of models. By applying the corresponding first plurality of responses to the first set of models 218, the at the respective client device 300 and/or the computer system 200 determines a baseline resilience of the respective subject at the respective first time. Said otherwise, in some such embodiments, the method 400 provides a quantitative measure for the baseline resilience responsive to the respective subject responding to the first questionnaire 208-1. However, the present disclosure is not limited thereto.

In some embodiments, the first set of models 218 includes one model 218, two or more models 218, three or more models 218, four or more models 218, five or more models 218, six or more models 218, seven or more models 218, eight or more models 210, fifteen or more models 218, or the like.

In some embodiments, the first set of models 218 includes a first cohort-based questionnaire response normalization model 218-1. In some embodiments, the first cohort-based questionnaire response normalization model 218-1 spans at least 3 cohorts, at least 5 cohorts, at least 10 cohorts, at least 20 cohorts, at least 50 cohorts, or the like. Accordingly, in some such embodiments, the computer system 200 utilizes the first cohort-based questionnaire response normalization model 218-1 for determining a quantification of a degradation effect exhibited by the respective subject when the respective subject is exposed to adversity in relation to a pool of cohorts. In some embodiments, the first cohort-based questionnaire response normalization model 218-1 is a T-norm model 218, in which a first order moment and a second moment order of two or more cohorts is utilized to normalize the corresponding first plurality of responses of the respective subject. Additional details and information regarding the first cohort-based questionnaire response normalization model 218-1 can be found at Sun et al., 2012, “Cohort Normalization Based Sparse Representation for Undersampled Face Recognition,” Proceedings of the Workshop on Face Analysis: The Intersection of Computer Vision and Human Perception as part of the 11th Asian Conference on Computer Vision, pg. 1, which is hereby incorporated by reference in its entirety.

In some embodiments, a respective model 218 in the first set of models 218 is implemented as an artificial intelligence engine. For instance, in some embodiments, the model 218 includes one or more gradient boosting models 218, one or more random forest models 218, one or more neural network (NN) models 218, one or more regression models 218, one or more Naïve Bayes models 218, one or more machine learning algorithms (MLA) 218, or a combination thereof. In some embodiments, an MLA or a NN model 218 is trained from a training data set that includes one or more features identified from a data set. MLAs models 218 include supervised algorithms (such as algorithms where the features/classifications in the data set are annotated) using linear regression, logistic regression, decision trees, classification and regression trees, Naïve Bayes, nearest neighbor clustering; unsupervised algorithms (such as algorithms where no features/classification in the data set are annotated a priori), such as means clustering, principal component analysis, random forest, adaptive boosting; and semi-supervised algorithms (such as algorithms where an incomplete number of features/classifications in the data set are annotated) using generative approach (such as a mixture of Gaussian distributions, mixture of multinomial distributions, hidden Markov models), low density separation, graph-based approaches (such as minimum cut, harmonic function, manifold regularization, etc.), heuristic approaches, or support vector machines.

Neural network models 218 include conditional random fields models 218, convolutional neural network (CNN) models 218, attention based neural network models 218, deep learning models 218, long short term memory network model 218, or other neural models 218.

While MLA and neural networks models 218 identify distinct approaches to machine learning, the terms may be used interchangeably herein. Thus, in some embodiments, a reference to MLA model 218 includes a corresponding NN model 218 or a reference to NN may include a corresponding MLA unless explicitly stated otherwise. In some embodiments, the training of a respective model includes providing one or more optimized datasets, labeling these features as they occur (e.g., in user profile 222 records), and training the MLA to predict or classify based on new inputs. Artificial NNs are efficient computing models which have shown their strengths in solving hard problems in artificial intelligence. For instance, artificial NNs have also been shown to be universal approximators, that is, they can represent a wide variety of functions when given appropriate parameters.

One of skill in the art will readily appreciate other models 218 that are applicable to the systems and methods of the present disclosure. In some embodiments, the systems and methods of the present disclosure utilize more than one model 218 to provide an evaluation (e.g., arrive at an evaluation given one or more inputs), such as an identity of a respective objective feedback to provide to the respective subject responsive to the corresponding first plurality of responses of the respective subject. For instance, in some embodiments, each respective model 218 arrives at a corresponding evaluation (e.g., a determination of a best objective feedback 214) when provided a respective data set in the form of a respective questionnaire response. Accordingly, in some embodiments, each respective model 218 independently arrives at a result and then the result of each respective model 218 is collectively verified through a comparison or amalgamation of the models 218. From this, a cumulative result is provided by the models 218. However, the present disclosure is not limited thereto.

In some embodiments, a respective model 218 is tasked with performing a corresponding activity. As a non-limiting example, in some embodiments, the task performed by the respective model 218 includes, but is not limited to, generating a first questionnaire 208-1 (e.g., block 420 of FIG. 4A), determining a baseline resilience of the respective subject (e.g., block 440 of FIG. 4C), generating a corresponding interactive digital journal associated with the respective subject (e.g., block 448 of FIG. 4C), exposing the respective subject to the corresponding intervention treatment regimen (e.g., block 454 of FIG. 4D), charting the progress of the respective subject (e.g., block 454 of FIG. 4D), updating the respective user profile 222 associated with the respective subject (e.g., block 490 of FIG. 4F and/or block 496 of FIG. 4F), determining if the respective subject is deemed to have completed a curriculum in a corresponding plurality of curriculum hosted by the computer system 200 (e.g., block 492 of FIG. 4F), or any combination thereof.

In some embodiments, each respective model 218 of the present disclosure makes use of 10 or more parameters, 100 or more parameters, 1000 or more parameters, 10,000 or more parameters, or 100,000 or more parameters. In some embodiments, each respective model 218 of the present disclosure cannot be mentally performed.

Block 442. Referring to block 442, in some embodiments, the applying the corresponding first plurality of responses to the first set of models 218 includes applying each respective response in the corresponding first plurality of responses to a corresponding model 218 in the first set of models 218. Said otherwise, in some embodiments, the corresponding model 218 is tasked with evaluating a particular response in the corresponding first plurality of responses. For instance, in some embodiments, one or more responses in the corresponding first plurality of responses from a WHO-5 questionnaire 208, a B-IPF questionnaire, a PSS-4, or a combination thereof (e.g., first questionnaire 208-1) is evaluated by a mixed linear model 218 in the first set of models 218.

Block 444. Referring to block 444, in some embodiments, the first set of models 218 includes a second Likert scale model 218-2. In some such embodiments, the second Likert scale model 218-2 is utilized by the computer system 200 in order to determine a quantifiable measure of an attitude of the respective subject. Accordingly, the second Likert scale model 218-2 is utilized by the computer system 200 to evaluate the respective subject (e.g., understand about one or more opinions and/or one or more perceptions of the respective subject) in relation to a respective latent interest (e.g., a phenomenon of interest).

Block 446. Referring to block 446, in some embodiments, the baseline resilience of the respective subject includes an indication of an adverse condition affecting the respective subject. For instance, in some such embodiments, the adverse condition affecting the subject includes a clinically diagnosed mental or psychiatric disorder or sub-clinically diagnosed mental or psychiatric disorder. In some embodiments, the adverse condition affecting the subject is an anxiety disorder, a mood disorder (e.g., depressive disorder), an addiction disorder (e.g., alcohol disorder), or the like.

Block 448. Referring to block 448, the method 400 includes generating a corresponding interactive digital journal associated with the respective subject for the respective subject. Accordingly, the computer system 200 allows the respective subject access to a corresponding intervention treatment regimen by way of the corresponding interactive digital journal. In this way, the corresponding intervention treatment regimen is designed to improve the resilience of the respective subject.

In some embodiments, the corresponding interactive digital journal is configured, at least in part, to chart a progress of the respective subject through the intervention treatment regimen. For instance, in some embodiments, embodiments, the charting the progress of the respective subject through the intervention treatment regimen allows for treatment of the respective subject through a visualization of a user interface displayed on a display of the respective client device, which depicts the progress of the respective subject over a period of time for a plurality of results from two or more questionnaires 208 (e.g., first questionnaire 208-1 of block 420 of FIG. 4A) that suggest one or more particular etiologies that should be addressed by the respective subject to improve a mental health state of the respective subject. However, the present disclosure is not limited thereto.

In some embodiments, the charting of the progress is provided by capturing a corresponding subjective narration by the respective subject of an exposure to the corresponding intervention treatment regimen. In some such embodiments, the corresponding subjective narration is captured in electronic form at the respective client device 300.

In some embodiments, the charting of the progress is provided by capturing a corresponding objective feedback (e.g., first objective feedback 214-1 of FIG. 2B). In some embodiments, the corresponding objective feedback 214 is captured based on a corresponding second plurality of responses of the respective subject. For instance, in some embodiments, the objective feedback 214 is associated with an optimism of the respective subject, an ability of the respective subject to facing one or more fears of the respective subject, a value of a social support structure of the respective subject, a physical health of the respective subject, an altruism of the respective subject, a cognitive flexibility of the respective subject, an emotional flexibility of the respective subject, a moral compass of the respective subject, an indication and/or an identity of one or more role models of the respective subject, a measure of brain fitness of the respective subject, or a combination thereof. Referring briefly to FIGS. 7D through 7E, in some embodiments, the objective feedback 214 includes displaying informational text to through the display of the respective client device 300 in order to inform the respective subject about an objective aspect of the respective subject, such as a indication of depression exhibited by the respective subject or a recommendation (e.g., instruction to interact with reality) for the respective subject to improve the objective aspect of the respective subject, such as talking to a doctor being doing vigorous exercise.

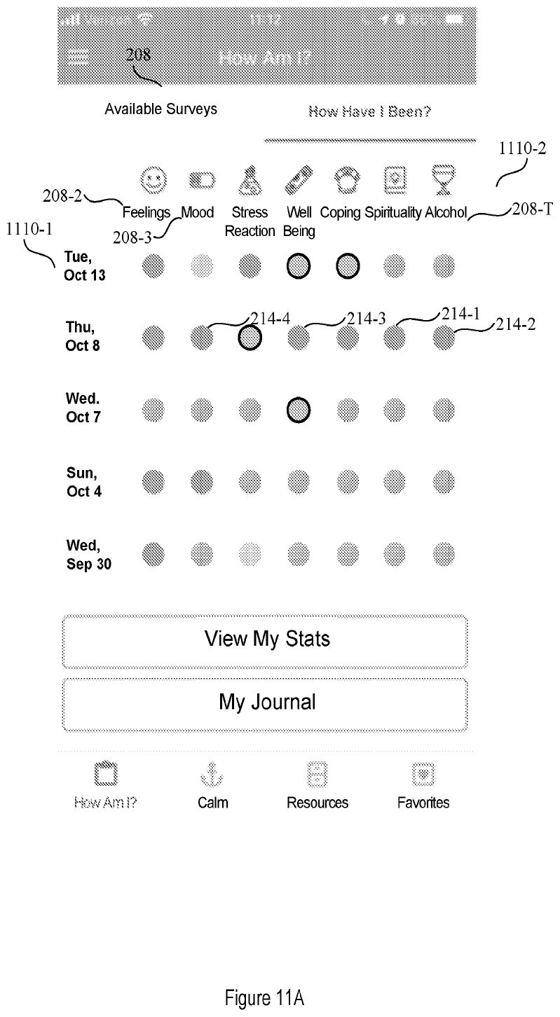

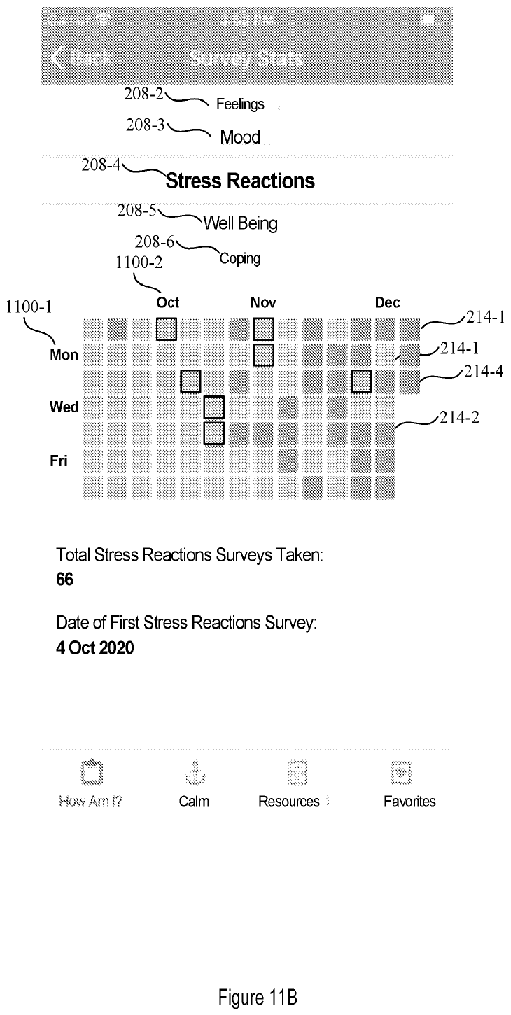

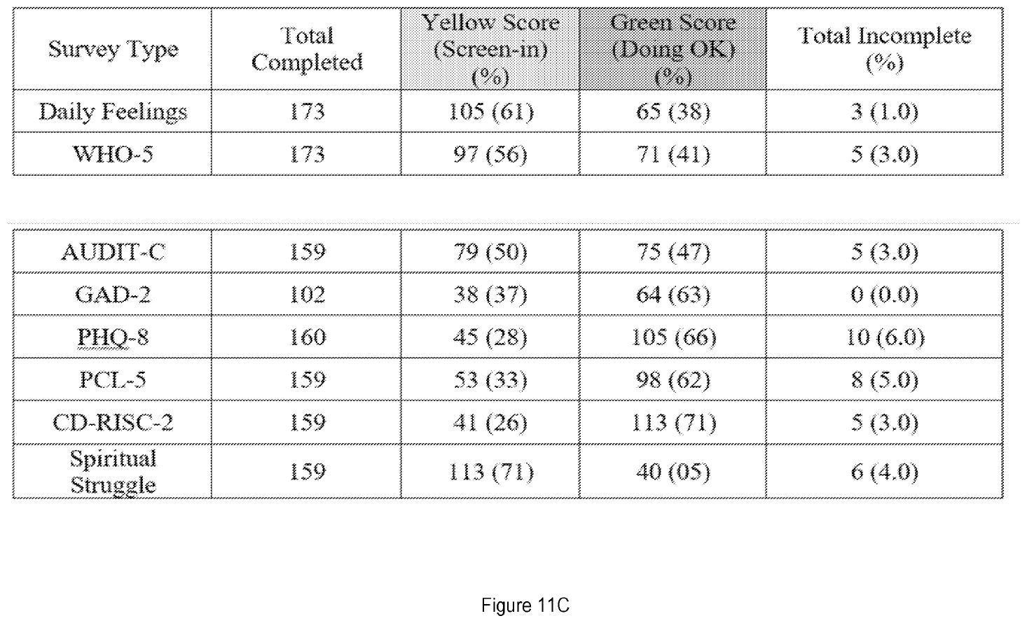

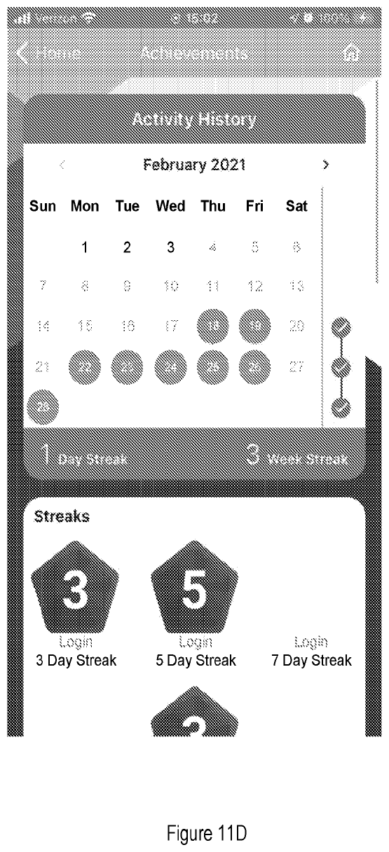

In some embodiments, the objective feedback 214 is captured by modifying an aspect of a graphical icon presented through the user interface of the respective client device 300. For instance, in some such embodiments, the graphical icon is associated with a respective data plot of the graphical chart 1100, and the aspect of the graphical icon includes a color of the graphical icon, an emoticon of the graphical icon, a shape of the graphical icon, a transparency of the graphical icon, and the like. For instance, referring briefly to FIGS. 11A and 11B, in some such embodiments, the color aspect of the graphical icon changes from a first color for no response by the respective subject, a second color for a good or positive response by the respective subject (e.g., improved resilience exhibited by the respective subject), a third color for a negative or bad response by the respective subject (e.g., diminished resilience exhibited by the respective subject), or a combination thereof. Accordingly, by viewing the graphical chart and the variety of modifications of the aspect of the graphical icon that is captured by the objective feedback 214, the respective subject is allowed to visualize trends in the behavior and/or resilience of the respective subject over the period of time. Moreover, by having the objective feedback 214 captured through the graphical chart 1100 of the digital journal, the respective use is able to view an overall analysis of the responses to a plurality of questionnaires 208 provided to the respective subject. Accordingly, responses that indicate improvements in the health of the respective subject are graphically modified by the systems and methods of the present disclosure to contour with a different color mapping schemes that indicate unique ranges within various curriculum. For instance, in some embodiments, a first data plot associated with a first objective feedback is indicated in a first (e.g., neutral) color to identify that the respective expressed concern about this resilience factorization, or indicated in a second color to identify a different expression by the respective subject when responding to a corresponding question and/or questionnaire. However, the present disclosure is not limited thereto.

In some embodiments, by capturing the objective feedback 214 and presenting the objective feedback to the respective user, the present disclosure provides an improved user-computer interface (e.g., user interface for display on the display of the respective client device 300) that is utilized by the systems and methods of the present disclosure for identifying appropriate resources that is a respective objective feedback and presenting these appropriate resources of the objective feedback to the respective subject. From this presentation of the objective feedback 214 that includes appropriate peer-reviewed resources, the user interface of the present disclosure is utilized to treat a specific etiology for a mental health crisis exhibited by the respective subject, such as by implementing the corresponding intervention treatment regimen for the respective subject.

Furthermore, in some such embodiments, the respective subject has a reduced mental burden since the user interface requires fewer user inputs (e.g., directional inputs, navigations, keystrokes, etc.) that are required to access the information provided by the objective feedback if the respective user sought this information elsewhere. Accordingly, the user interface of the present disclosure improves an efficiency and/or battery life of the respective client device 300 since the user interface requires fewer user inputs to capture the objective feedback 214, and, therefore, consumes less energy of the battery.

In some embodiments, the second plurality of responses is provided by the respective subject when provided (e.g., in electronic form using communication network 106 of FIG. 1) a second questionnaire 208-2. In some embodiments, the second plurality of responses is provided by the respective subject during the exposure to the respective intervention treatment regimen. Accordingly, the corresponding objective feedback 214 is configured to arouse a behavioral change in the respective subject.

Block 450. Referring to block 450, in some embodiments, the corresponding intervention treatment regimen for the respective subject is self-guided by the respective subject. For instance, in some embodiments, the method 400 is performed for the respective subject without human interface except for the respective subject. Said otherwise, in some such embodiments, the method 400 is performed without any contact between the respective subject and another subject, such as a therapist or coach. However, the present disclosure is not limited thereto. For instance, in some embodiments, the therapist or coach assist or supports the respective subject by providing the digital media that is authored, at least in part, by the therapist or coach.

Block 452. Referring to block 452, in some embodiments, the second questionnaire 208-2 is a subset of the first questionnaire 208-1. For instance, in some embodiments, the second questionnaire 208-2 includes a first subset of undecided responses of the respective subject from the first questionnaire 208-1 and/or a second subset of responses for a particular category or aspect of the resilience of the subject, such as a respective curriculum in the corresponding plurality of curriculum.

Block 454. Referring to block 454 of FIG. 4D, the method 400 includes exposing the respective subject to the corresponding intervention treatment regimen. In some embodiments, the method 400 exposes the respective subject to the corresponding intervention treatment regimen by display, through a user interface 376 on the display 380 of the respective client device 300, objective feedback 214 that is particular to the respective subject based on one or more responses provided by the respective subject one a respective questionnaire 208.

The corresponding intervention treatment regimen includes a corresponding plurality of curricula. Each respective curriculum in the corresponding plurality of curricula: is associated with a unique factorization of a respective resilience of the respective subject. In some embodiments, the respective resilience of the respective subject includes two or more factorizations, 5 or more factorizations, 10 or more factorizations, or fifty or more factorizations. Additional details and information regarding the corresponding plurality of curricula can be found at Herman et al., 2001, “What is Resilience?,” The Canadian Journal of Psychiatry, 56(5), pg. 258; Southwick et al., 2018, “Resilience: The Science of Mastering Life's Greatest Challenges,” Cambridge University Press, print, each of which is hereby incorporated by reference in its entirety.

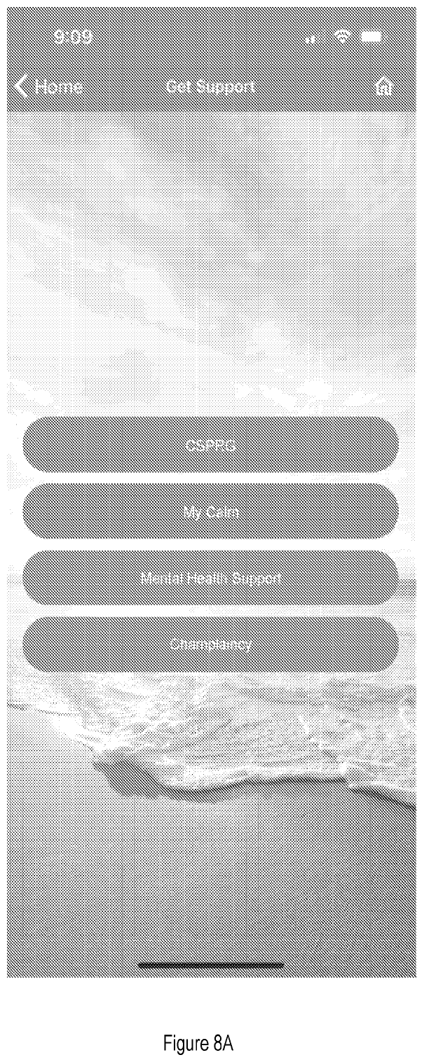

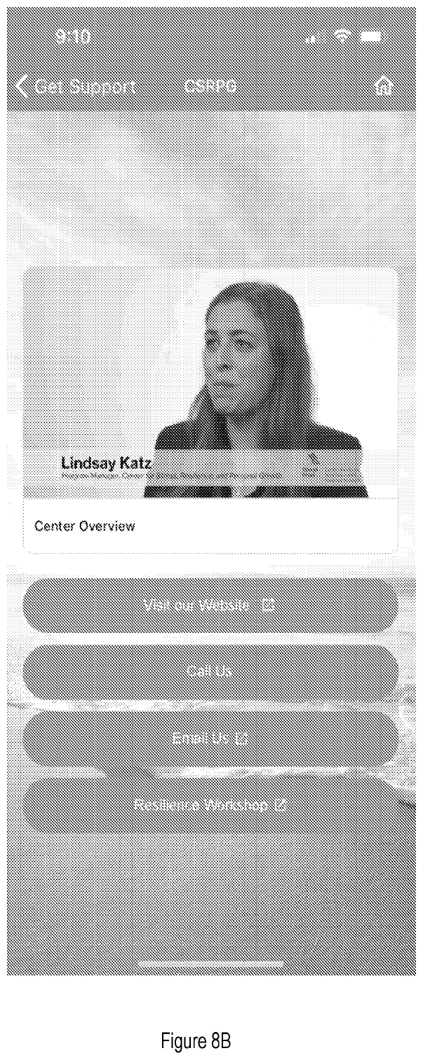

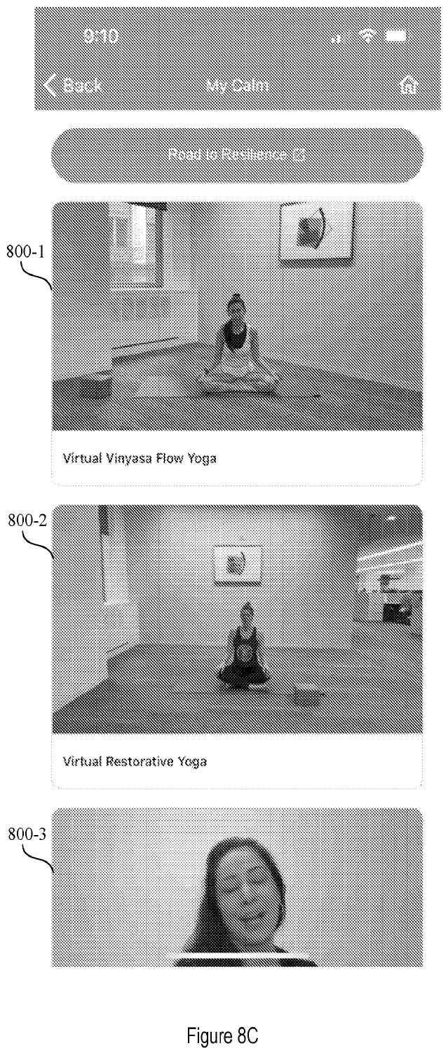

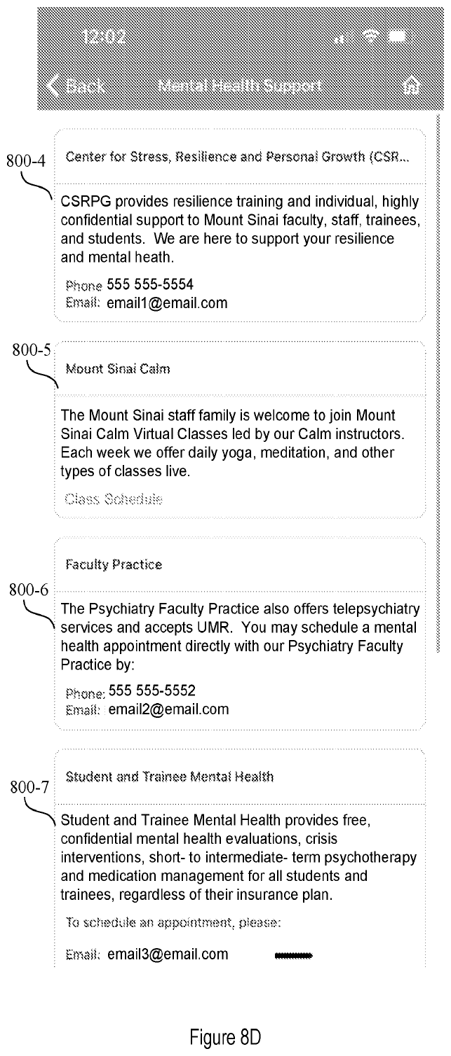

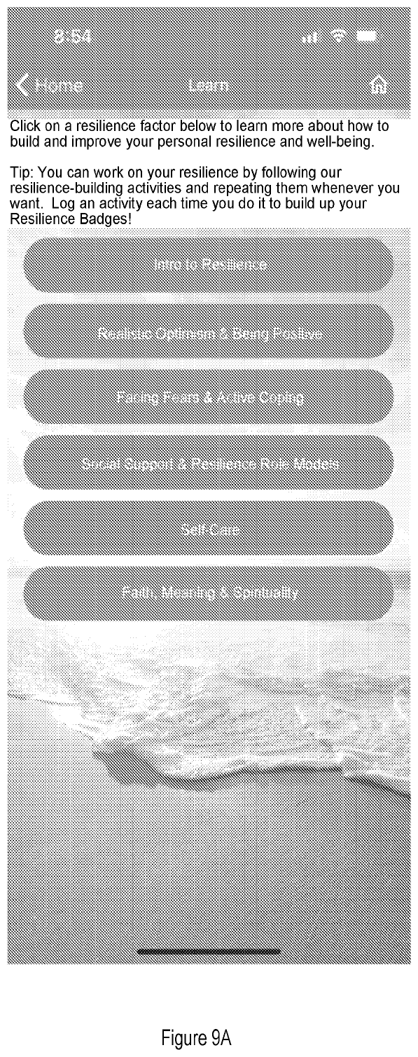

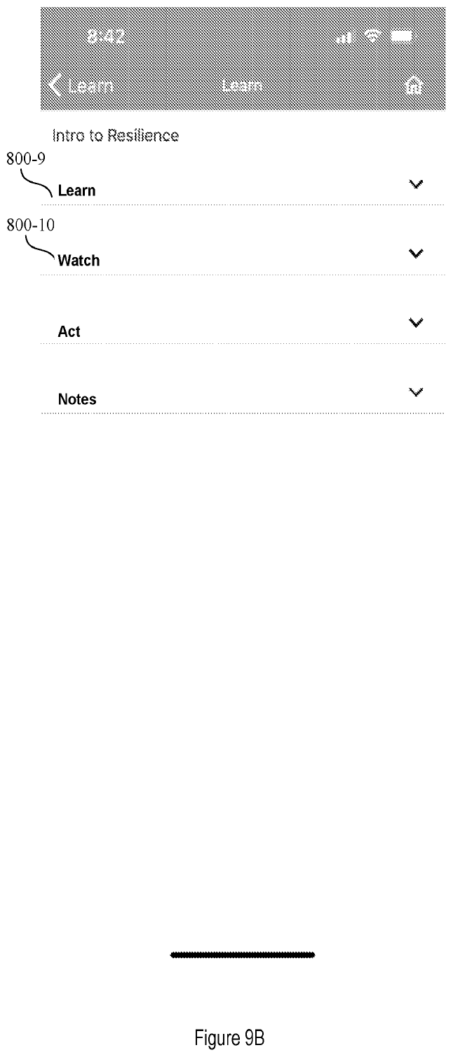

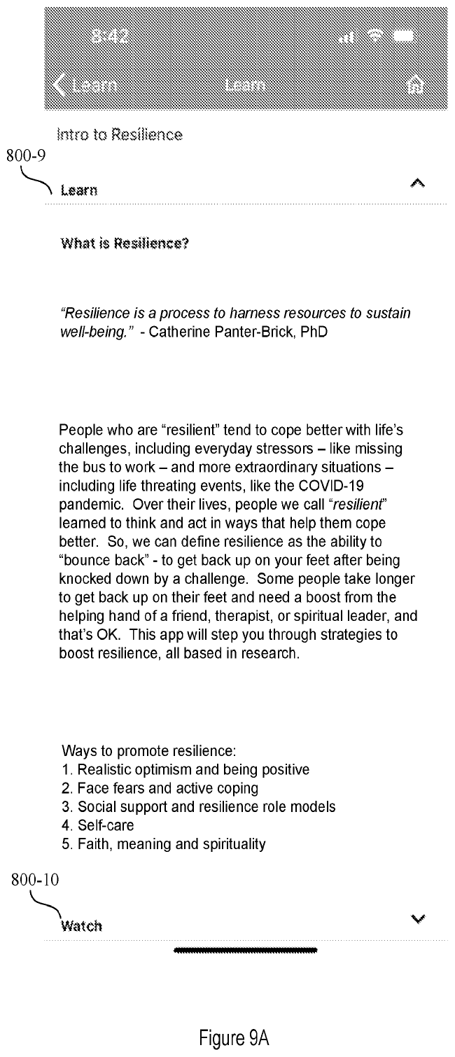

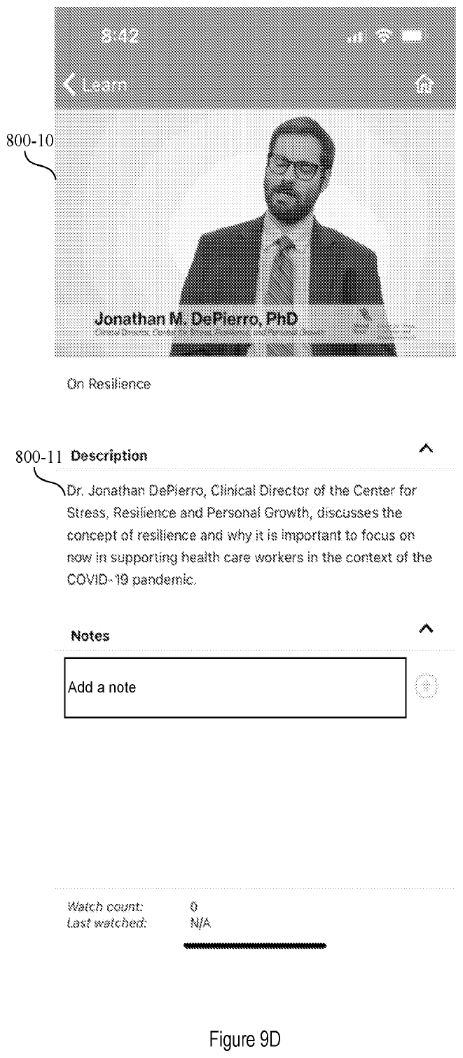

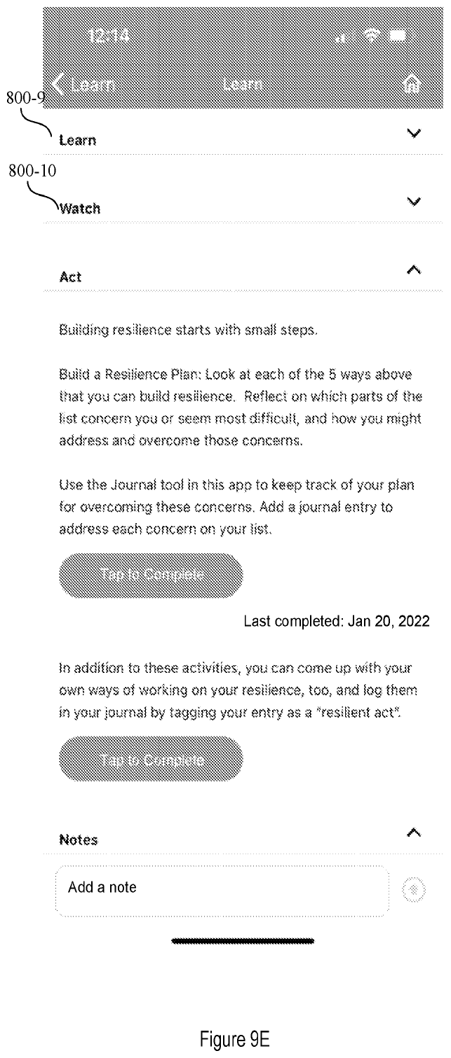

Furthermore, in some embodiments, each respective curriculum includes a unique compilation of digital media (e.g., digital media 800 of FIG. 8A, digital media 800 of FIG. 9C, digital media 800 of FIG. 9D, etc.). Each respective digital media in the unique compilation of digital media is configured to improve an ability of the respective subject associated with the unique factorization of the respective resilience. For instance, in some embodiments, the ability of the respective subject is improved by written (e.g., textual) digital media and/or video digital media that includes psychoeducation content about a specific resilience aspect, behavioral activation tips for the respective subject to promote resilience-building behavior, homework assignments (e.g., instructions to interact with reality) related to the resilience aspect, or a combination thereof.

In some embodiments, the unique compilation of digital media includes 2 items of digital media, at least 3 items of digital media, at least 10 items of digital media, or at least 15 items of digital media.

Additionally, in some embodiments, referring briefly to FIG. 9E, each respective curriculum includes a corresponding set of instructions for the respective subject to interact with reality. In some embodiments, the interactions with reality by the respective subject include providing one or more inputs through the client device 300. However, the present disclosure is not limited thereto.

In some embodiments, the corresponding plurality of curricula includes a first curriculum associated with a cognitive appraisal of the respective subject. For instance, referring briefly to FIG. 9A, a user interface 900-1 displays a visualization of a corresponding plurality of curricula including the first curriculum that is associated with the cognitive appraisal of the respective subject by addressing realistic optimism as staying positive for the respective subject. the first curriculum includes a corresponding set of instructions including a first instruction for the respective subject to write a plurality of items that the respective subject is grateful for.

In some embodiments, the corresponding plurality of curricula includes a second curriculum associated with confronting a first locus of fear experienced by the respective subject. For instance, referring briefly to FIG. 9A, a user interface 900-1 displays a visualization of the corresponding plurality of curricula including the second curriculum that is associated with the confronting a first locus of fear experienced by the respective subject by addressing fears faced by the respective subject and/or active coping mechanisms.

In some embodiments, the corresponding plurality of curricula includes a third curriculum associated with an environmental reality system determined, at least in part, by the respective subject. For instance, referring briefly to FIG. 9A, a user interface 900-1 displays a visualization of the corresponding plurality of curricula including the third curriculum that is associated with environmental reality system determined by the respective subject by addressing social support circles and/or one or more subjects that the respective subject idealizes as a role model.

In some embodiments, the corresponding plurality of curricula includes a fourth curriculum associated with a plurality of biological factors of the respective subject. For instance, referring briefly to FIG. 9A, a user interface 900-1 displays a visualization of the corresponding plurality of curricula including the fourth curriculum that is associated with environmental reality system determined by the respective subject by addressing self-caring habits of the respective user, such as sleep and/or exercise, or one or more similar self-caring hygienic habits of the respective subject.

In some embodiments, the corresponding plurality of curricula includes a fifth curriculum associated with confronting a second locus of purpose of the respective subject. However, the present disclosure is not limited thereto. For instance, referring briefly to FIG. 9A, a user interface 900-1 displays a visualization of the corresponding plurality of curricula including the fifth curriculum that is associated with environmental reality system determined by the respective subject by addressing a faith, meaning, and/or spirituality associated with the respective subject. Additional details and information regarding confronting the second locus of purpose of the respective subject can be found at Steinhauser et al., 2017, “State of the Science of Spirituality and Palliative Care Research Part I: Definitions, Measurements, and Outcomes,” Journal of Pain and Symptom Management,” 54(3), pg. 428, which is hereby incorporated by reference in its entirety.

In some embodiments, the corresponding set of instructions includes a first instruction for opening scripture and/or prayer (e.g., one scripture example is provided for each instruction), a second instruction for defining and understanding a respective factorization aspect of resilience, a third instruction for reflecting on the experiences of the respective subject with each factorized aspect of resilience, a fourth instruction for discussing how to foster that resilience factorized aspect through different activities in reality, a fifth instruction for asking a different subject (e.g., in reality) for assistance with a problem, and the like. However, the present disclosure is not limited thereto.

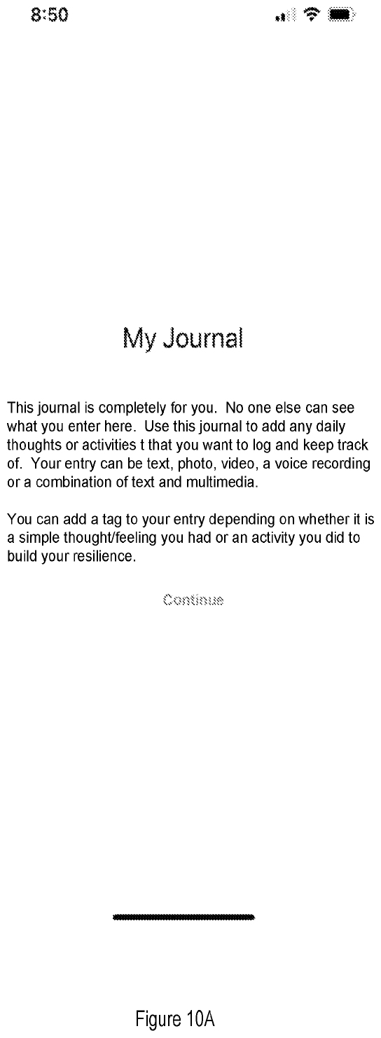

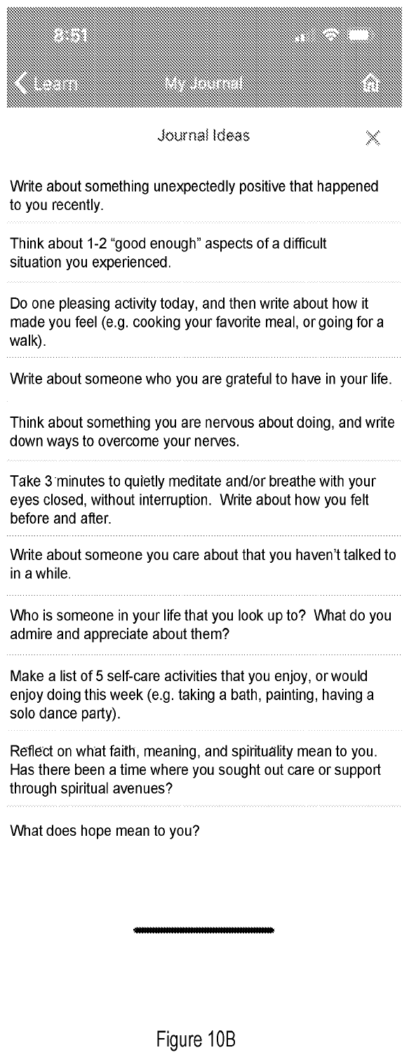

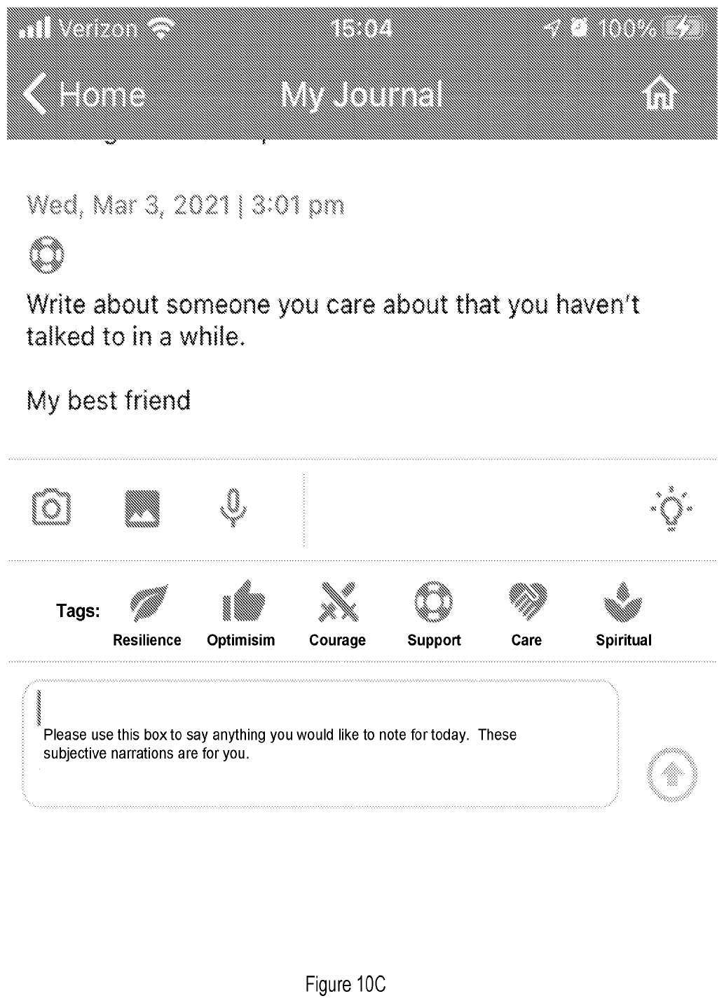

Block 456. Referring to block 456, in some embodiments, the corresponding set of instructions for the respective subject to interact with reality includes a first instruction for the respective subject to input a respective subjective narration by the respective subject. For instance, referring briefly to FIG. 9E, in some embodiments, the first instruction is for the respective subject to input a respective subjective narration by the respective subject to keep track of a plan by the respective subject to overcome a list of concerns and/or an entry of a resilient act by the respective subject in reality. However, the present disclosure is not limited thereto. For instance, referring briefly to FIG. 10B, in some embodiments, the corresponding set of instructions is visually listed for the respective subject.

Block 458. Referring to block 458, in some embodiments, the subjective narration by the respective subject of the exposure to the corresponding intervention treatment regimen includes a duration of a disruptive event producing adversity, a severity of the disruptive event producing adversity, an outcome of disruptive event producing adversity, or a combination thereof.

Block 460. Referring to block 460, in some embodiments, the subjective narration by the respective subject of the exposure to the corresponding intervention treatment regimen includes a duration of a disruptive event producing adversity, a severity of the disruptive event producing adversity, an outcome of disruptive event producing adversity, or a combination thereof.

Block 462. Referring to block 462, in some embodiments, the unique compilation of digital media includes a first set of textual digital media. For instance, referring briefly to FIG. 9A, a first digital media 800-1 is displayed on a user interface of the respective client device 300, which includes information (e.g., objective feedback 214) for consumption by the respective subject,

Block 464. Referring to block 464, in some embodiments, the first set of textual digital media includes a lecture text portion and/or an activity text portion, such as a first activity based on the one or more instructions for the respective subject to interact with reality. For instance, referring briefly to FIGS. 9B through 9E, a user interface for displaying a unique compilation of digital media associated with a first curriculum is provided. In some embodiments, the first set of textual digital media includes the lecture text portion that provides educational information and materials for consumption by the respective subject, which, when consumed by the respective subject, provide treatment for the respective subject.

Block 466. Referring to block 466, in some embodiments, the unique compilation of digital media includes a second set of video digital media of various media content (e.g., .avi, .mkv, .mp4, etc.).

In some embodiments, the unique compilation of digital medica includes a third set of image digital media of various media content (e.g., .gif, .jpeg, .pdf, .png, etc.).

Block 468. Referring to block 468, in some embodiments, the second set of video digital media includes a lecture by a medical practitioner credentialed to improve the ability of the respective subject associated with the unique factorization of the resilience. As a non-limiting example, referring briefly to FIGS. 8C and 9D, in some embodiments, the computer system 200 provides a library video digital media (e.g., “My Calm” of FIG. 8C), such as a first plurality of video digital media associated with a relaxation subject matter. In some embodiments, the library of video digital media includes a first video digital media that is publicly available (e.g., published, acquired through the communication network 106 of FIG. 1, etc.) and/or a second video digital media that is private. In some embodiments, each respective video digital media ranges in topic and/or duration so the respective subject is allowed to select the respective video digital media that works best for the respective subject in the moment. For example, a first subject choose a short, 2-minute video digital media if the first subject is on break during the workday, but prefer to engage with a longer, more involved yoga video digital media in the evening or to start the day. However, the present disclosure is not limited thereto.

In some embodiments, the method 400 allows the respective subject to select the respective digital media as a favorite. In some embodiments, the favorited digital media is retained in a corresponding user profile (e.g., first user profile 222-1 of FIG. 2B) associated with the respective subject for easily accessibility by the respective subject when needed at a future time.

Block 470. Referring to block 470 of FIG. 4E, in some embodiments, the charting the progress of the respective subject further includes providing a graphical chart (e.g., first graphical chart 1100 of FIG. 11A, second graphical chart 1100 of FIG. 11B, etc.). For instance, referring briefly to FIGS. 11A through 11D, one or more graphical charts is provided that display the progress of the respective subject. Accordingly, the graphical chart includes a first axis that includes units by a first segmentation (e.g., first axis 1110-1 of FIG. 11A, first axis 1110-1 of FIG. 11B, etc.). In some embodiments, the first segmentation is based on a period of time, such as a contiguous rolling period of time that encompasses about 30 days, about 60 days, about 90 days, about 360 days, or the like.

In some embodiments, the graphical chart includes a second axis that includes units by a second segmentation. In some embodiments, the second segmentation is based on a respective set of responses provided by the respective subject.

Accordingly, each respective axis of the graphical chart is a feature on the respective graphical chart that extends in a single direction (e.g., a line, a ray, etc.) along which information is presented and/or conveyed through and defines a dimension (e.g., boundary) of the respective graphical chart. That is, in some such embodiments, a position (e.g., location) of a feature (e.g., data plot) on the respective graphical chart is used to convey information to the respective subject with respect to the respective axis. From this, each respective axis allows the respective subject to visualize relative positions between data plots that populate an area formed by two or more axes on the respective graphical chart. Moreover, in some embodiments, the first axis is orthogon to the second axis. In this way, the respective graphical chart illustrates one or more quadrant of a two-dimensional coordinate plane that is formed by the first axis and the second axis.

In some embodiments, a respective axis is displayed as a visible feature of the respective graphical chart. For instance, in some such embodiments, a respective second axis is displayed as a bar shaped feature of the respective graphical chart. In some embodiments, the respective axis is hidden feature of the respective graphical chart.

Accordingly, in some such embodiments, the graphical chart includes one or more data plots. Each respective data plot in the one or more data plots represents a corresponding set of responses in a corresponding second plurality of responses of the respective subject when provided the second questionnaire during the period of time. Furthermore, each respective data plot in the one or more data plots populates a unique location within the area formed by the first axis and the second axis. Said otherwise, in some such embodiments, a first data plot in the one or more data plots does not overlap a second data plot in the one or more data plots. Accordingly, the method 400 allows the respective subject to visualize progress through the corresponding interactive intervention regimen by viewing the positioning of the one or more data plots displayed through the respective graphical chart. However, the present disclosure is not limited thereto.

In some embodiments, the respective client device 300 is restricted to displaying one respective graphical chart at a time (e.g., limited by a display configuration of the respective graphical chart and/or the client application 306 of the client device 300), allowing the respective subject to focus and comprehend the information provided by the respective graphical chart without distraction of a second graphical chart. However, the present disclosure is not limited thereto.

In some embodiments, the corresponding interactive digital journal includes at least one respective graphical chart for each respective curriculum in the corresponding plurality of curricula. In some embodiments, the corresponding interactive digital journal includes at least one respective graphical chart for each unique factorization of a respective resilience of the respective subject. Furthermore, in some embodiments, the corresponding interactive digital journal includes at least one respective graphical chart for each respective questionnaire 208 in a plurality of questionnaires 208 hosted by the computer system 200.

Block 472. Referring to block 472, the first segmentation and/or the second segmentation is a linear segmentation or a logarithmic segmentation. For instance, in some embodiments, the first segmentation and/or the second segmentation is a monotonic function relation (e.g., square root function relation or logarithmic function relation). For instance, with the square root function relation, if a respective first ratio segmentation is 2:1, then a respective second ratio of a first feature of a first data plot and a second feature of a second data plot is 4:1. Furthermore, in some embodiments, when the segmentation is the logarithmic function relation, the first ratio is proportional to log(N), with any logarithmic base, proportional to log(N+c), or proportional to c+log(N), where c is a constant. In some embodiments, the constant c is a positive number, such as 1, and N is a positive integer. Also, in some embodiments, a blended function is used by the computer system 200. For instance, in some embodiments, the segmentation of a respective axis of the respective graphical chart is different for different ranges of N. As a non-limiting example, in some embodiments, the segmentation of the respective axis is: (i) proportional or approximately proportional to N for values of N that are below a first threshold value of N; (ii) proportional, or approximately proportional, to the square-root of N over an intermediate range of values of N that is greater than or equal to the first threshold value of N and less than or equal to a second threshold value of N; and (iii) proportional, or approximately proportional, to log(N) above the second threshold value of N. However, the present disclosure is not limited thereto.

Block 474. Referring to block 474, an initial time of the period of time is the first time. Said otherwise, in some embodiments, an initial point (e.g., origin) of the respective graphical chart is the first time when the respective subject provided the corresponding first plurality of responses from the first questionnaire 208-1 (e.g., block 420 of FIG. 4A).

Block 476. Referring to block 476, in some embodiments, each respective data plot in the one or more data plots is associated with a corresponding graphical icon in accordance with a status of the corresponding set of responses. In some embodiments, the corresponding graphical icon represents a respective parameter or characteristics of the respective resilience of the respective subject that is represented by a corresponding data plot. Furthermore, in some embodiments, a respective graphical icon 616 is a group of pixels represented in one or more colors that is different from a color of an adjacent pixel. In some embodiments, a respective graphical icon 616 is a text label (e.g., graphical icon 616-3 of FIG. 6A including text label “[1]” that represents a prodrug pharmaceutical composition class). In some embodiments, a respective graphical icon 616 is an image (e.g., graphical icon 616-1 of FIG. 6B including an image of a scale).

Block 478. Referring to block 478, in some embodiments, the first curriculum associated with the cognitive appraisal of the respective subject is configured in accordance with a respective life-stage of the respective subject. For instance, in some embodiments, the respective life-stage of the respective subject is partitioned into two or more cohorts based on a biological maturity and/or mental maturity of the respective subject.

Block 480. Referring to block 480, in some embodiments, the first curriculum associated with the cognitive appraisal of the respective subject includes determining an intellectual function score of the respective subject.

Block 482. Referring to block 482, in some embodiments, the first curriculum associated with the cognitive appraisal of the respective subject includes determining a cognitive flexibility score of the respective subject. For instance, in some embodiments, the determination of the cognitive flexibility score of the respective subject includes determining a status of an intentional change in a behavior of the respective subject, determining a status of an incremental change in the behavior of the respective subject, determining a status of an evolutionary change in the behavior of the respective subject, determining a status of an unintentional change in the behavior of the respective subject, determining a status of a transformative change in the behavior of the respective subject, or a combination thereof. Additional details and information regarding the determination of the cognitive flexibility score of the respective subject can be found at Joseph et al., 2006, “Growth following adversity: Theoretical perspectives and implications for clinical practice,” Clinical psychology review, 26(8), pg. 1041, which is hereby incorporated by reference in its entirety.

Block 484. Referring to block 484, in some embodiments, the second curriculum associated with confronting the first locus of fear experienced by the respective subject includes suppressing a hypothalamic-pituitary-adrenal (HPA) response of the respective subject, which includes a negative feedback regulation of the HPA response based on psychosocial stressors. Additional details and information regarding the HPA response of the respective subject can be found at Gunnar et al., 2006, “Bringing Basic Research on Early Experience and Stress Neurobiology to Bear on Preventive Interventions for Neglected and Maltreated Children,” Development and Psychopathology, 18(3), pg. 651; Meaney, M., 2001, “Maternal Care, Gene Expression, and the Transmission of Individual Differences in Stress Reactivity Across Generations,” Annual Review of Neuroscience, 24(1), pg. 1161, each of which is hereby incorporated by reference in its entirety.

Block 486. Referring to block 486 of FIG. 4F, in some embodiments, the fourth curriculum associated with the plurality of biological factors of the respective subject includes a change in brain size of the respective subject, a change in sensitivity of neural receptors of the respective subject, a change in neurotransmitter synthetic and/or uptake, or a combination thereof. In some embodiments, the change in brain size of the respective subject is determined, at least in part, based on an indication of an induction of a neural plate of the respective subject, a localized proliferation of a plurality of viable cells in different regions of the brain of the respective subject, a migration of the plurality of cells from a first region of the brain in which the plurality of cells is generated to the places where the cells ultimately reside, an aggregation of cells to form identifiable parts of the brain of the respective subject, a differentiation (e.g., expression) of one or more immature neurons by the respective subject, a formation of one or more axonal pathways and one or more synaptic connections with other neurons and an onset of physiological function exhibited by the respective subject, a selective death of one or more predetermined cell types of the respective subject, an elimination of some of the one or more connections that were initially formed and a stabilization of others of the one or more connections of the brain of the respective subject, or a combination thereof. Additional details and information regarding the change in brain size of the respective subject can be found at CHAPTER, G. O. T., FURTHERING, A. P. T., & NEUROBIOLOGICAL, I. I. T, 2006, “The Developing Brain and Neural Plasticity: Implications for Normality, Psychopathology, and Resilience,” Developmental Psychopathology, 2, pg. 1, which is hereby incorporated by reference in its entirety.

Block 488. Referring to block 488, in some embodiments, the fifth curriculum associated with confronting the second locus of purpose of the respective subject includes determining a first score of an importance of the second locus of the respective subject and/or determining a coping classification of the respective subject.

Block 490. Referring to block 490, the method 400 includes updating a respective user profile associated with the respective subject in response to the receiving the first alert.

In some embodiments, the respective user profile associated with the respective subject is utilized to determine and retain a plurality of key performance indicators (KPIs). Each respective KPI in the plurality of KPI's is utilized to measure an aspect of success of the interactive treatment regimen and/or a level of engagement of the respective subject. In some embodiments, the plurality of KPIs include a first KPI associated with a total number of first questionnaires 208-1 submitted by the respective subject, a total number of second questionnaires 208-2 submitted by the respective subject (e.g., post-baseline engagement by the respective subject), general engagement metrics (e.g., number of openings of the client application 306 by the respective subject within a period of time). However, the present disclosure is not limited thereto.

Block 492. Referring to block 492, the method 400 includes receiving a respective second alert, in electronic form at the computer system, from the respective client device associated with the respective subject that the respective subject has concluded the corresponding method, thereby updating, in accordance with a determination that the respective subject is deemed to have completed a curriculum in the corresponding plurality of curricula, the objective feedback of the corresponding interactive digital journal.

Block 494. Referring to block 494, in some embodiments, the determination the respective subject is deemed to complete the curriculum in the corresponding plurality of curricula includes determining whether one or more instructions in the corresponding set of instructions for the respective subject to interact with reality is deemed completed.

Block 496. Referring to block 496, the method 400 includes updating the respective user profile associated with the respective subject in response to the receiving the second alert, thereby implementing the corresponding intervention treatment regimen for the respective subject.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a non-transitory computer readable storage medium. For instance, the computer program product could contain the program modules shown in any combination of FIGS. 2A, 2B, 3, 5, 6A, 6B, 7A, 7B, 7C, 7D, 7E, 7F, 7G, 8A, 8B, 8C, 8D, 9A, 9B, 9C, 9D, 9E, 10A, 10B, 10C, 11A, 11B, 11C, and 11D. These program modules can be stored on a CD-ROM, DVD, magnetic disk storage product, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed is:

1. A computer system comprising:
one or more processing units and a memory coupled to at least one processing unit of the one or more processing units, the memory storing at least one program for execution by the at least one processing unit, the at least one program comprising one or more instructions for:
for each respective subject in a plurality of subjects, wherein the plurality of subjects comprises more than 100 subjects:
(A) receiving a respective first alert, in electronic form at the computer system, from a respective client device associated with a respective subject that the respective subject has initiated a corresponding method, the corresponding method comprising:
receiving, in electronic form at the respective client device, a corresponding first plurality of responses of the respective subject when provided a first questionnaire at a respective first time, wherein each respective response in the corresponding first plurality of responses is associated with a corresponding value, and wherein the corresponding first plurality of responses comprises:
a corresponding first set of responses associated with an ecological mood of the respective subject,
a corresponding second set of responses indicative of whether the respective subject exhibits a depressive disorder or an anxiety disorder,
a corresponding third set of responses indicative of a stress reaction of the respective subject,
a corresponding fourth set of responses signifying a self-reported resilience of the respective subject,
a corresponding fifth set of responses indicative of a general wellness state of the respective subject,
a corresponding sixth set of responses indicative of whether the respective subject exhibits an alcohol disorder, and
a corresponding seventh set of responses that characterizes a state of spirituality of the respective subject;
applying, at the respective client device or the computer system, the corresponding first plurality of responses to a first set of models, thereby determining a baseline resilience of the respective subject at the respective first time, wherein the first set of models comprises a first cohort-based questionnaire response normalization model;
generating, for the respective subject, a corresponding interactive digital journal associated with the respective subject, thereby allowing the respective subject access to a corresponding intervention treatment regimen by the corresponding interactive digital journal, wherein the corresponding intervention treatment regimen is designed to improve the resilience of the respective subject, and wherein the corresponding interactive digital journal is configured, at least in part, to chart a progress of the respective subject through the intervention treatment regimen relative to the baseline resilience by capturing:
a corresponding subjective narration, in electronic form at the respective client device, by the respective subject of an exposure to the corresponding intervention treatment regimen, and
a corresponding objective feedback based on a corresponding second plurality of responses of the respective subject when provided, in electronic form, a second questionnaire during the exposure to the respective intervention treatment regimen, wherein the corresponding objective feedback is configured to arouse a behavioral change in the respective subject;
exposing the respective subject to the corresponding intervention treatment regimen, wherein the corresponding intervention treatment regimen comprises a corresponding plurality of curricula, wherein each respective curriculum in the corresponding plurality of curricula:
is associated with a unique factorization of a respective resilience of the respective subject and,
comprises (i) a unique compilation of digital media configured to improve an ability of the respective subject associated with the unique factorization of the respective resilience of the respective subject, and (ii) a corresponding set of instructions for the respective subject to interact with reality, and
wherein the corresponding plurality of curricula comprises:
a first curriculum associated with a cognitive appraisal of the respective subject,
a second curriculum associated with confronting a first locus of fear experienced by the respective subject,
a third curriculum associated with an environmental reality system determined, at least in part, by the respective subject,
a fourth curriculum associated with a plurality of biological factors of the respective subject, and
a fifth curriculum associated with confronting a second locus of purpose of the respective subject; and
(B) updating a respective user profile associated with the respective subject in response to the receiving (A);
(C) receiving a respective second alert, in electronic form at the computer system, from the respective client device associated with the respective subject that the respective subject has concluded the corresponding method, thereby updating, in accordance with a determination that the respective subject is deemed to have completed a curriculum in the corresponding plurality of curricula, the objective feedback of the corresponding interactive digital journal; and
(D) updating the respective user profile associated with the respective subject in response to the receiving (C), thereby implementing the corresponding intervention treatment regimen for the respective subject.

2. The computer system of claim 1, wherein the intervention treatment regimen for the respective subject is self-guided by the respective subject.

3. The computer system according to either of claim 1 or 2, wherein the corresponding value is in a range of from 0 to 4, from 0 to 5, or from 0 to 10.

4. The computer system of either of claim 1 or 2, wherein the corresponding value is categorical.

5. The computer system of any preceding claim, wherein the first questionnaire comprises a first set of questions indicative of whether the respective subject exhibits a depressive indicator.

6. The computer system of claim 5, wherein the depressive indicator comprises a sleep quality rating of the respective subject, a mood rating of the respective subject, an anxiety rating of the respective subject, or a combination thereof.

7. The computer system of any preceding claim, wherein the first questionnaire comprises a second set of questions configured to identify a generalized anxiety disorder exhibited by the respective subject.

8. The computer system of any preceding claim, wherein the corresponding first plurality of responses comprises an eighth set of responses associated with a demographic status of the respective subject.

9. The computer system of claim 8, wherein the eighth set of responses comprises a gender of the respective subject, an age of the respective subject, a race of the respective subject, an employment status of the respective subject, an education status of the respective subject, a military service status of the respective subject, an income status of the respective subject, an adverse childhood experience status of the respective subject, or a combination thereof.

10. The computer system of any preceding claim, wherein the first questionnaire is substantially identical to the second questionnaire.

11. The computer system according to any one of claims 1-9, wherein the second questionnaire is a subset of the first questionnaire.

12. The computer system of any preceding claim, wherein the first questionnaire comprises from about 20 questions to about 75 questions.

13. The computer system of any preceding claim, wherein the applying the corresponding first plurality of responses to the first set of models comprises applying each respective response in the corresponding first plurality of responses to a corresponding model in the first set of models.

14. The computer system of any preceding claim, wherein the first set of models comprises a second Likert scale model.

15. The computer system of any preceding claim, wherein the baseline resilience of the respective subject comprises an indication of an adverse condition affecting the respective subject.

16. The computer system of any preceding claim, wherein the corresponding set of instructions for the respective subject to interact with reality comprises a first instruction for the respective subject to input a respective subjective narration by the respective subject.

17. The computer system of any preceding claim, wherein the subjective narration by the respective subject of the exposure to the intervention treatment regimen comprises a duration of a disruptive event producing adversity, a severity of the disruptive event producing adversity, an outcome of disruptive event producing adversity, or a combination thereof.

18. The computer system of any preceding claim, wherein the unique compilation of digital media comprises a first set of textual digital media.

19. The computer system of claim 18, wherein the first set of textual digital media comprises a lecture text portion and/or an activity text portion.

20. The computer system of any preceding claim, wherein the unique compilation of digital media comprises a second set of video digital media.

21. The computer system of claim 20, wherein the second set of video digital media comprises a lecture by a medical practitioner credentialed to improve the ability of the respective subject associated with the unique factorization of the resilience.

22. The computer system of any preceding claim, wherein the charting the progress of the respective subject further comprises providing a graphical chart comprising:
a first axis by a first segmentation based on a period of time,
a second axis by a second segmentation based on a respective set of responses the corresponding first plurality of responses comprises provided by the respective subject, and
one or more data plots, wherein each respective data plot in the one or more data plots (i) represents a corresponding set of responses in a corresponding second plurality of responses of the respective subject when provided the second questionnaire during the period of time, and (ii) populates a unique location within an area formed by the first axis and the second axis.

23. The computer system of claim 22, wherein the first segmentation and/or the second segmentation is a linear segmentation or a logarithmic segmentation.

24. The computer system according to either of claim 22 or 23, wherein an initial time of the period of time is the first time.

25. The computer system according to any one of claims 22-24, wherein each respective data plot in the one or more data plots is associated with a corresponding graphical icon in accordance with a status of the corresponding set of responses.

26. The computer system of any preceding claim, wherein the first curriculum associated with the cognitive appraisal of the respective subject is configured in accordance with a respective life-stage of the respective subject.

27. The computer system of any preceding claim, wherein the first curriculum associated with the cognitive appraisal of the respective subject comprises determining an intellectual function score of the respective subject.

28. The computer system of any preceding claim, wherein the first curriculum associated with the cognitive appraisal of the respective subject comprises determining a cognitive flexibility score of the respective subject.

29. The computer system of any preceding claim, wherein the second curriculum associated with confronting the first locus of fear experienced by the respective subject comprises suppressing a hypothalamic-pituitary-adrenal response of the respective subject.

30. The computer system of any preceding claim, wherein the fourth curriculum associated with the plurality of biological factors of the respective subject comprises a change in brain size of the respective subject, a change in sensitivity of neural receptors of the respective subject, a change in neurotransmitter synthetic and/or uptake, or a combination thereof.

31. The computer system of any preceding claim, wherein the fifth curriculum associated with confronting the second locus of purpose of the respective subject comprises determining a first score of an importance of the second locus of the respective subject and/or determining a coping classification of the respective subject.

32. The computer system of any preceding claim, wherein the determination the respective subject is deemed to complete the curriculum in the corresponding plurality of curricula comprises determining whether one or more instructions in the corresponding set of instructions for the respective subject to interact with reality is deemed complete.

33. The computer system of any preceding claim, wherein the computer system performs the (A) receiving the respective first alert for 250 hundred or more subjects, a thousand or more subjects, or fifty thousand or more subjects.

34. The computer system of any preceding claim, wherein the respective client device is a mobile phone.

35. The computer system of claim 34, wherein the mobile phone comprises a touch-sensitive display.

36. The computer system of claim 35, wherein the mobile phone further comprises a power system for powering the touch-sensitive display, a processing unit, and one or more intensity sensors that receive a user input.

37. The computer system of claim 36, wherein the power system comprises a rechargeable battery.

38. The computer system according to either of claim 36 or 37, wherein the user input comprises a directional user input.

39. The computer system according to any one of claims 36-38, wherein the user input is for providing a respective response of the subject.

40. A computer readable storage medium storing one or more programs, the one or more programs comprising instructions, which when executed by an electronic server device with one or more processors and a memory cause the electronic server device to perform a method comprising:
for each respective subject in a plurality of subjects, wherein the plurality of subjects comprises more than 100 subjects:
(A) receiving a respective first alert, in electronic form at the electronic server device, from a respective client device associated with a respective subject that the respective subject has initiated a corresponding method, the corresponding method comprising:
receiving, in electronic form at the respective client device, a corresponding first plurality of responses of the respective subject when provided a first questionnaire at a respective first time, wherein each respective response in the corresponding first plurality of responses is associated with a corresponding value, and wherein the corresponding first plurality of responses comprises:
a corresponding first set of responses associated with an ecological mood of the respective subject,
a corresponding second set of responses indicative of whether the respective subject exhibits a depressive disorder or an anxiety disorder,
a corresponding third set of responses indicative of a stress reaction of the respective subject,
a corresponding fourth set of responses signifying a self-reported resilience of the respective subject,
a corresponding fifth set of responses indicative of a general wellness state of the respective subject,
a corresponding sixth set of responses indicative of whether the respective subject exhibits an alcohol disorder, and
a corresponding seventh set of responses that characterizes a state of spirituality of the respective subject;
applying, at the respective client device or the electronic server device, the corresponding first plurality of responses to a first set of models, thereby determining a baseline resilience of the respective subject at the respective first time, wherein the first set of models comprises a first cohort-based questionnaire response normalization model;
generating, for the respective subject, a corresponding interactive digital journal associated with the respective subject, thereby allowing the respective subject access to a corresponding intervention treatment regimen by the corresponding interactive digital journal, wherein the corresponding intervention treatment regimen is designed to improve the resilience of the respective subject, and wherein the corresponding interactive digital journal is configured, at least in part, to chart a progress of the respective subject through the intervention treatment regimen relative to the baseline resilience by capturing:
a corresponding subjective narration, in electronic form at the respective client device, by the respective subject of an exposure to the corresponding intervention treatment regimen, and
a corresponding objective feedback based on a corresponding second plurality of responses of the respective subject when provided, in electronic form, a second questionnaire during the exposure to the respective intervention treatment regimen, wherein the corresponding objective feedback is configured to arouse a behavioral change in the respective subject;
exposing the respective subject to the corresponding intervention treatment regimen, wherein the corresponding intervention treatment regimen comprises a corresponding plurality of curricula, wherein each respective curriculum in the corresponding plurality of curricula:
is associated with a unique factorization of a respective resilience of the respective subject and,
comprises (i) a unique compilation of digital media configured to improve an ability of the respective subject associated with the unique factorization of the respective resilience, and (ii) a corresponding set of instructions for the respective subject to interact with reality, and
wherein the corresponding plurality of curricula comprises:
a first curriculum associated with a cognitive appraisal of the respective subject,
a second curriculum associated with confronting a first locus of fear experienced by the respective subject,
a third curriculum associated with an environmental reality system determined, at least in part, by the respective subject,
a fourth curriculum associated with a plurality of biological factors of the respective subject, and
a fifth curriculum associated with confronting a second locus of purpose of the respective subject; and
(B) updating a respective user profile associated with the respective subject in response to the receiving (A);
(C) receiving a respective second alert, in electronic form at the electronic server device, from the respective client device associated with the respective subject that the respective subject has concluded the corresponding method, thereby updating, in accordance with a determination that the respective subject is deemed to have completed a curriculum in the corresponding plurality of curricula, the objective feedback of the corresponding interactive digital journal; and
(D) updating the respective user profile associated with the respective subject in response to the receiving (C), thereby implementing the corresponding intervention treatment regimen for the respective subject.

41. A client device for providing a user interface on a touch-sensitive display for identifying an objective feedback and viewing the objective feedback to treat an etiology for a mental health condition of a subject associated with the client device, the client device comprising the touch sensitive display, one or more processing units, and a memory storing one or more programs for executing by the one or more processing units, wherein the one or more programs comprise one or more instructions for providing the user interface comprising:
a first user interface screen comprising a first affordance region representing an interactive questionnaire, wherein the interactive questionnaire comprises a plurality of selectable responses for a corresponding plurality of prompts; and
a second user interface screen comprising a second affordance region representing the objective feedback, wherein the second affordance region is configured to display a notification that corresponds to the respective objective feedback.

42. The client device of claim 41, wherein the user interface facilitates receiving, in electronic form at the client device, a first plurality of responses of the subject from the interactive questionnaire at a respective first time, wherein each respective response in the first plurality of responses is associated with a corresponding value, and wherein the first plurality of responses comprises:
a first set of responses associated with an ecological mood of the subject,
a second set of responses indicative of whether the subject exhibits a depressive disorder or an anxiety disorder,
a third set of responses indicative of a stress reaction of the subject,
a fourth set of responses signifying a self-reported resilience of the subject,
a fifth set of responses indicative of a general wellness state of the subject,
a sixth set of responses indicative of whether the subject exhibits an alcohol disorder, and
a seventh set of responses that characterizes a state of spirituality of the subject.

43. The client device of claim 42, wherein the one or more programs further comprise one or more instructions for:
applying the first plurality of responses to a first set of models, thereby determining a baseline resilience of the subject at the respective first time, wherein the first set of models comprises a first cohort-based questionnaire response normalization model; and
generating, for the subject, an interactive digital journal associated with the subject, thereby allowing the subject access to an intervention treatment regimen by the interactive digital journal, wherein the intervention treatment regimen is designed to improve the resilience of the subject, and wherein the interactive digital journal is configured, at least in part, to chart a progress, through the second user interface, of the subject through the intervention treatment regimen relative to the baseline resilience by capturing:
a subjective narration, in electronic form at the client device, by the subject of an exposure to the intervention treatment regimen, and
the objective feedback, wherein the objective feedback is based on a second plurality of responses of the subject when provided, in electronic form, a second questionnaire during the exposure to the intervention treatment regimen, wherein the objective feedback is configured to arouse a behavioral change in the subject.

44. The client device of claim 43, further comprising exposing the subject to the intervention treatment regimen, wherein the intervention treatment regimen comprises a plurality of curricula, wherein each curriculum in the plurality of curricula:
is associated with a unique factorization of a resilience of the subject and,
comprises (i) a compilation of digital media configured to improve an ability of the subject associated with the unique factorization of the resilience of the respective subject, and (ii) a set of instructions for the subject to interact with reality, and wherein the corresponding plurality of curricula comprises:
a first curriculum associated with a cognitive appraisal of the subject,
a second curriculum associated with confronting a first locus of fear experienced by the subject,
a third curriculum associated with an environmental reality system determined, at least in part, by the subject,
a fourth curriculum associated with a plurality of biological factors of the subject, and
a fifth curriculum associated with confronting a second locus of purpose of the subject.

45. The client device of claim 43 or 44, wherein the intervention treatment regimen for the respective subject is self-guided by the respective subject.

46. The client device according to any one of claims 42-45, wherein the corresponding value is in a range of from 0 to 4, from 0 to 5, or from 0 to 10.

47. The client device according to any one of claims 42-45, wherein the corresponding value is categorical.

48. The client device of according to any one of claims 42-47, wherein the interactive questionnaire comprises a first set of questions indicative of whether the subject exhibits a depressive indicator.

49. The client device of claim 48, wherein the depressive indicator comprises a sleep quality rating of the respective subject, a mood rating of the respective subject, an anxiety rating of the respective subject, or a combination thereof.

50. The client device according to any one of claims 42-49, wherein the interactive questionnaire comprises a second set of questions configured to identify a generalized anxiety disorder exhibited by the respective subject.

51. The client device according to any one of claims 42-50, wherein the corresponding first plurality of responses comprises an eighth set of responses associated with a demographic status of the respective subject.

52. The client device of claim 8, wherein the eighth set of responses comprises a gender of the respective subject, an age of the respective subject, a race of the respective subject, an employment status of the respective subject, an education status of the respective subject, a military service status of the respective subject, an income status of the respective subject, an adverse childhood experience status of the respective subject, or a combination thereof.

53. The client device according to any one of claims 43-52, wherein the interactive questionnaire is substantially identical to the second questionnaire.

54. The client device according to any one of claims 43-52, wherein the second questionnaire is a subset of the interactive questionnaire.

55. The client device according to any one of claims 41-54, wherein the interactive questionnaire comprises from about 20 questions to about 75 questions.

56. The client device of claim 43, wherein the applying the first plurality of responses to the first set of models comprises applying each respective response in the first plurality of responses to a corresponding model in the first set of models.

57. The client device of claim 56, wherein the first set of models comprises a second Likert scale model.

58. The client device according to any one of claims 43-57, wherein the baseline resilience of the respective subject comprises an indication of an adverse condition affecting the respective subject.

59. The client device according to any one of claims 44-58, wherein the corresponding set of instructions for the subject to interact with reality comprises a first instruction for the subject to input the subjective narration by the subject.

60. The client device of claim 59, wherein the subjective narration by the subject of the exposure to the intervention treatment regimen comprises a duration of a disruptive event producing adversity, a severity of the disruptive event producing adversity, an outcome of disruptive event producing adversity, or a combination thereof.

61. The client device according to any one of claims 44-60, wherein the compilation of digital media comprises a first set of textual digital media.

62. The client device of claim 18, wherein the first set of textual digital media comprises a lecture text portion and/or an activity text portion.

63. The client device according to any one of claims 44-60, wherein the unique compilation of digital media comprises a second set of video digital media.

64. The client device of claim 63, wherein the second set of video digital media comprises a lecture by a medical practitioner credentialed to improve the ability of the subject associated with the unique factorization of the resilience.

65. The client device according to any one of claims 43-60, wherein the charting the progress of the subject further comprises providing a graphical chart comprising:
a first axis by a first segmentation based on a period of time,
a second axis by a second segmentation based on a respective set of responses in the first plurality of responses provided by the subject, and
one or more data plots, wherein each respective data plot in the one or more data plots (i) represents a corresponding set of responses in a corresponding second plurality of responses of the subject when provided the second questionnaire during the period of time, and (ii) populates a unique location within an area formed by the first axis and the second axis.

66. The client device of claim 65, wherein the first segmentation and/or the second segmentation is a linear segmentation or a logarithmic segmentation.

67. The client device according to either of claim 65 or 66, wherein an initial time of the period of time is the first time.

68. The client device according to any one of claims 65-67, wherein each respective data plot in the one or more data plots is associated with a corresponding graphical icon in accordance with a status of the respective set of responses.

69. The client device according to any one of claims 44-68, wherein the first curriculum associated with the cognitive appraisal of the subject is configured in accordance with a respective life-stage of the subject.

70. The client device according to any one of claims 44-68, wherein the first curriculum associated with the cognitive appraisal of the subject comprises determining an intellectual function score of the subject.

71. The client device according to any one of claims 44-68, wherein the first curriculum associated with the cognitive appraisal of the subject comprises determining a cognitive flexibility score of the subject.

72. The client device according to any one of claims 44-71, wherein the second curriculum associated with confronting the first locus of fear experienced by the subject comprises suppressing a hypothalamic-pituitary-adrenal response of the subject.

73. The client device according to any one of claims 44-72, wherein the fourth curriculum associated with the plurality of biological factors of the subject comprises a change in brain size of the subject, a change in sensitivity of neural receptors of the subject, a change in neurotransmitter synthetic and/or uptake, or a combination thereof.

74. The client device according to any one of claims 44-73, wherein the fifth curriculum associated with confronting the second locus of purpose of the subject comprises determining a first score of an importance of the second locus of the subject and/or determining a coping classification of the subject.

75. The client device according to any one of claims 44-73, wherein the client device is a mobile phone.

76. A method for treating a respective etiology for a mental health condition, the method comprising:
at a server device comprising one or more processing units and a memory coupled to at least one processing unit of the one or more processing units, the memory storing at least one program for execution by the at least one processing unit, the at least one program comprising one or more instructions for, for each respective subject in a plurality of subjects, wherein the plurality of subjects comprises more than 100 subjects:
(A) determining an identification of a respective etiology of the respective subject by applying a corresponding plurality of responses of the respective subject to a first set of models; and
(B) displaying, on a display of a client device associated with the respective subject or the server device, a progress of the respective subject when exposed to a corresponding intervention treatment regimen for the respective subject, thereby visualizing a progress of the respective subject indicated by the corresponding plurality of responses that suggests the respective etiology that should be addressed by the respective subject to improve the mental health condition of the respective subject.
